SEQUENCE LISTING (74 sequences)

SEQ ID NO: 1 - RNA, 14 nt, Synthetic construct
  source 1..14: mol_type = other RNA
  modified_base 8: mod_base = m5c
agttangcacccaa

SEQ ID NO: 2 - RNA, 18 nt, synthetic construct
  source 1..18: mol_type = other RNA
  modified_base 5: mod_base = m5c
  modified_base 4: mod_base = m5c
  modified_base 14: mod_base = m5c
  modified_base 17: mod_base = m5c
gtgccataggaatcatct

SEQ ID NO: 3 - RNA, 18 nt, Synthetic construct
  source 1..18: mol_type = other RNA
  modified_base 4: mod_base = m5c
  modified_base 5: mod_base = m5c
  modified_base 14: mod_base = m5c
gtgccataggaatcatct

SEQ ID NO: 4 - RNA, 18 nt, synthetic construct
  source 1..18: mol_type = other RNA
  modified_base 5: mod_base = m5c
  modified_base 6: mod_base = m5c
  modified_base 15: mod_base = m5c
  modified_base 18: mod_base = m5c
ggtgccataggaatcatc

SEQ ID NO: 5 - RNA, 18 nt, Synthetic construct
  source 1..18: mol_type = other RNA
  modified_base 6: mod_base = m5c
  modified_base 7: mod_base = m5c
  modified_base 16: mod_base = m5c
tggtgccataggaatcat

SEQ ID NO: 6 - RNA, 18 nt, Synthetic construct
  source 1..18: mol_type = other RNA
  modified_base 1: mod_base = m5c
  modified_base 7: mod_base = m5c
  modified_base 8: mod_base = m5c
  modified_base 17: mod_base = m5c
ctggtgccataggaatca

SEQ ID NO: 7 - RNA, 18 nt, Synthetic construct
  source 1..18: mol_type = other RNA
  modified_base 2: mod_base = m5c
  modified_base 8: mod_base = m5c
  modified_base 9: mod_base = m5c
  modified_base 18: mod_base = m5c
gctggtgccataggaatc

SEQ ID NO: 8 - RNA, 18 nt, Synthetic construct
  source 1..18: mol_type = other RNA
  modified_base 3: mod_base = m5c
  modified_base 9: mod_base = m5c
  modified_base 10: mod_base = m5c
ggctggtgccataggaat

SEQ ID NO: 9 - RNA, 18 nt, Synthetic construct
  source 1..18: mol_type = other RNA
  modified_base 10: mod_base = m5c
  modified_base 4: mod_base = m5c
  modified_base 11: mod_base = m5c
tggctggtgccataggaa

SEQ ID NO: 10 - RNA, 18 nt, Synthetic construct
  source 1..18: mol_type = other RNA
  modified_base 1: mod_base = m5c
  modified_base 5: mod_base = m5c
  modified_base 11: mod_base = m5c
  modified_base 12: mod_base = m5c
ctggctggtgccatagga

SEQ ID NO: 11 - RNA, 18 nt, Synthetic construct
  source 1..18: mol_type = other RNA
  modified_base 1: mod_base = m5c
  modified_base 2: mod_base = m5c
  modified_base 6: mod_base = m5c
  modified_base 12: mod_base = m5c
  modified_base 13: mod_base = m5c
cctggctggtgccatagg

SEQ ID NO: 12 - RNA, 18 nt, Synthetic construct
  source 1..18: mol_type = other RNA
  modified_base 2: mod_base = m5c
  modified_base 3: mod_base = m5c
  modified_base 7: mod_base = m5c
  modified_base 13: mod_base = m5c
  modified_base 14: mod_base = m5c
tcctggctggtgccatag

SEQ ID NO: 13 - RNA, 18 nt, Synthetic construct
  source 1..18: mol_type = other RNA
  modified_base 3: mod_base = m5c
  modified_base 4: mod_base = m5c
  modified_base 8: mod_base = m5c
  modified_base 14: mod_base = m5c
  modified_base 15: mod_base = m5c
ttcctggctggtgccata

SEQ ID NO: 14 - RNA, 18 nt, Synthetic construct
  source 1..18: mol_type = other RNA
  modified_base 4: mod_base = m5c
  modified_base 5: mod_base = m5c
  modified_base 9: mod_base = m5c
  modified_base 15: mod_base = m5c
  modified_base 16: mod_base = m5c
tttcctggctggtgccat

SEQ ID NO: 15 - RNA, 18 nt, Synthetic construct
  source 1..18: mol_type = other RNA
  modified_base 5: mod_base = m5c
  modified_base 6: mod_base = m5c
  modified_base 10: mod_base = m5c
  modified_base 16: mod_base = m5c
  modified_base 17: mod_base = m5c
ttttcctggctggtgcca

SEQ ID NO: 16 - RNA, 18 nt, Synthetic construct
  source 1..18: mol_type = other RNA
  modified_base 6: mod_base = m5c
  modified_base 7: mod_base = m5c
  modified_base 11: mod_base = m5c
  modified_base 17: mod_base = m5c
  modified_base 18: mod_base = m5c
tttttcctggctggtgcc

SEQ ID NO: 17 - RNA, 18 nt, Synthetic construct
  source 1..18: mol_type = other RNA
  modified_base 7: mod_base = m5c
  modified_base 8: mod_base = m5c
  modified_base 12: mod_base = m5c
  modified_base 18: mod_base = m5c
atttttcctggctggtgc

SEQ ID NO: 18 - RNA, 18 nt, Synthetic construct
  source 1..18: mol_type = other RNA
  modified_base 1: mod_base = m5c
  modified_base 8: mod_base = m5c
  modified_base 9: mod_base = m5c
  modified_base 13: mod_base = m5c
catttttcctggctggtg

SEQ ID NO: 19 - RNA, 18 nt, Synthetic construct
  source 1..18: mol_type = other RNA
  modified_base 1: mod_base = m5c
  modified_base 2: mod_base = m5c
  modified_base 9: mod_base = m5c
  modified_base 10: mod_base = m5c
  modified_base 14: mod_base = m5c
ccatttttcctggctggt

SEQ ID NO: 20 - RNA, 18 nt, Synthetic construct
  source 1..18: mol_type = other RNA
  modified_base 2: mod_base = m5c
  modified_base 3: mod_base = m5c
  modified_base 10: mod_base = m5c
  modified_base 11: mod_base = m5c
  modified_base 15: mod_base = m5c
gccatttttcctggctgg

SEQ ID NO: 21 - RNA, 18 nt, synthetic construct
  source 1..18: mol_type = other RNA
  modified_base 3: mod_base = m5c
  modified_base 4: mod_base = m5c
  modified_base 11: mod_base = m5c
  modified_base 12: mod_base = m5c
  modified_base 16: mod_base = m5c
tgccatttttcctggctg

SEQ ID NO: 22 - RNA, 18 nt, Synthetic construct
  source 1..18: mol_type = other RNA
  modified_base 1: mod_base = m5c
  modified_base 4: mod_base = m5c
  modified_base 5: mod_base = m5c
  modified_base 12: mod_base = m5c
  modified_base 13: mod_base = m5c
  modified_base 17: mod_base = m5c
ctgccatttttcctggct

SEQ ID NO: 23 - RNA, 18 nt, Synthetic construct
  source 1..18: mol_type = other RNA
  modified_base 2: mod_base = m5c
  modified_base 5: mod_base = m5c
  modified_base 6: mod_base = m5c
  modified_base 13: mod_base = m5c
  modified_base 14: mod_base = m5c
  modified_base 18: mod_base = m5c
gctgccatttttcctggc

SEQ ID NO: 24 - RNA, 18 nt, Synthetic construct
  source 1..18: mol_type = other RNA
  modified_base 3: mod_base = m5c
  modified_base 6: mod_base = m5c
  modified_base 7: mod_base = m5c
  modified_base 14: mod_base = m5c
  modified_base 15: mod_base = m5c
agctgccatttttcctgg

SEQ ID NO: 25 - RNA, 18 nt, Synthetic construct
  source 1..18: mol_type = other RNA
  modified_base 3: mod_base = m5c
  modified_base 6: mod_base = m5c
  modified_base 7: mod_base = m5c
  modified_base 14: mod_base = m5c
  modified_base 15: mod_base = m5c
agctgccatttttcctgg

SEQ ID NO: 26 - RNA, 18 nt, Synthetic construct
  source 1..18: mol_type = other RNA
  modified_base 4: mod_base = m5c
  modified_base 7: mod_base = m5c
  modified_base 8: mod_base = m5c
  modified_base 15: mod_base = m5c
  modified_base 16: mod_base = m5c
gagctgccatttttcctg

SEQ ID NO: 27 - RNA, 18 nt, Synthetic construct
  source 1..18: mol_type = other RNA
  modified_base 5: mod_base = m5c
  modified_base 8: mod_base = m5c
  modified_base 9: mod_base = m5c
  modified_base 16: mod_base = m5c
  modified_base 17: mod_base = m5c
agagctgccatttttcct

SEQ ID NO: 28 - RNA, 18 nt, Synthetic construct
  source 1..18: mol_type = other RNA
  modified_base 6: mod_base = m5c
  modified_base 9: mod_base = m5c
  modified_base 10: mod_base = m5c
  modified_base 17: mod_base = m5c
  modified_base 18: mod_base = m5c
aagagctgccatttttcc

SEQ ID NO: 29 - RNA, 18 nt, Synthetic construct
  source 1..18: mol_type = other RNA
  modified_base 7: mod_base = m5c
  modified_base 10: mod_base = m5c
  modified_base 11: mod_base = m5c
  modified_base 18: mod_base = m5c
taagagctgccatttttc

SEQ ID NO: 30 - RNA, 18 nt, Synthetic construct
  source 1..18: mol_type = other RNA
  modified_base 8: mod_base = m5c
  modified_base 11: mod_base = m5c
  modified_base 12: mod_base = m5c
ttaagagctgccattttt

SEQ ID NO: 31 - RNA, 18 nt, Synthetic construct
  source 1..18: mol_type = other RNA
  modified_base 9: mod_base = m5c
  modified_base 12: mod_base = m5c
  modified_base 13: mod_base = m5c
tttaagagctgccatttt

SEQ ID NO: 32 - RNA, 18 nt, Synthetic construct
  source 1..18: mol_type = other RNA
  modified_base 1: mod_base = m5c
  modified_base 10: mod_base = m5c
  modified_base 13: mod_base = m5c
  modified_base 14: mod_base = m5c
ctttaagagctgccattt

SEQ ID NO: 33 - RNA, 18 nt, synthetic construct
  source 1..18: mol_type = other RNA
  modified_base 1: mod_base = m5c
  modified_base 2: mod_base = m5c
  modified_base 11: mod_base = m5c
  modified_base 14: mod_base = m5c
  modified_base 15: mod_base = m5c
cctttaagagctgccatt

SEQ ID NO: 34 - RNA, 18 nt, Synthetic construct
  source 1..18: mol_type = other RNA
  modified_base 2: mod_base = m5c
  modified_base 3: mod_base = m5c
  modified_base 12: mod_base = m5c
  modified_base 15: mod_base = m5c
  modified_base 16: mod_base = m5c
tcctttaagagctgccat

SEQ ID NO: 35 - RNA, 14 nt, Homo sapiens
  source 1..14: mol_type = mRNA
ttgggtgcgtaact

SEQ ID NO: 36 - RNA, 18 nt, Homo sapiens
  source 1..18: mol_type = mRNA
agatgattcctatggcac

SEQ ID NO: 37 - RNA, 18 nt, Homo sapiens
  source 1..18: mol_type = mRNA
agatgattcctatggcac

SEQ ID NO: 38 - RNA, 18 nt, Homo sapiens
  source 1..18: mol_type = mRNA
gatgattcctatggcacc

SEQ ID NO: 39 - RNA, 18 nt, Homo sapiens
  source 1..18: mol_type = mRNA
atgattcctatggcacca

SEQ ID NO: 40 - RNA, 18 nt, Homo sapiens
  source 1..18: mol_type = mRNA
tgattcctatggcaccag

SEQ ID NO: 41 - RNA, 18 nt, Homo sapiens
  source 1..18: mol_type = mRNA
gattcctatggcaccagc

SEQ ID NO: 42 - RNA, 18 nt, Homo sapiens
  source 1..18: mol_type = mRNA
attcctatggcaccagcc

SEQ ID NO: 43 - RNA, 18 nt, Homo sapiens
  source 1..18: mol_type = mRNA
ttcctatggcaccagcca

SEQ ID NO: 44 - RNA, 18 nt, Homo sapiens
  source 1..18: mol_type = mRNA
tcctatggcaccagccag

SEQ ID NO: 45 - RNA, 18 nt, Homo sapiens
  source 1..18: mol_type = mRNA
cctatggcaccagccagg

SEQ ID NO: 46 - RNA, 18 nt, Homo sapiens
  source 1..18: mol_type = mRNA
ctatggcaccagccagga

SEQ ID NO: 47 - RNA, 18 nt, Homo sapiens
  source 1..18: mol_type = mRNA
tatggcaccagccaggaa

SEQ ID NO: 48 - RNA, 18 nt, Homo sapiens
  source 1..18: mol_type = mRNA
atggcaccagccaggaaa

SEQ ID NO: 49 - RNA, 18 nt, Homo sapiens
  source 1..18: mol_type = mRNA
tggcaccagccaggaaaa

SEQ ID NO: 50 - RNA, 18 nt, Homo sapiens
  source 1..18: mol_type = mRNA
ggcaccagccaggaaaaa

SEQ ID NO: 51 - RNA, 18 nt, Homo sapiens
  source 1..18: mol_type = mRNA
gcaccagccaggaaaaat

SEQ ID NO: 52 - RNA, 18 nt, Homo sapiens
  source 1..18: mol_type = mRNA
caccagccaggaaaaatg

SEQ ID NO: 53 - RNA, 18 nt, Homo sapiens
  source 1..18: mol_type = mRNA
accagccaggaaaaatgg

SEQ ID NO: 54 - RNA, 18 nt, Homo sapiens
  source 1..18: mol_type = mRNA
ccagccaggaaaaatggc

SEQ ID NO: 55 - RNA, 18 nt, Homo sapiens
  source 1..18: mol_type = mRNA
cagccaggaaaaatggca

SEQ ID NO: 56 - RNA, 18 nt, Homo sapiens
  source 1..18: mol_type = mRNA
agccaggaaaaatggcag

SEQ ID NO: 57 - RNA, 18 nt, Homo sapiens
  source 1..18: mol_type = mRNA
gccaggaaaaatggcagc

SEQ ID NO: 58 - RNA, 18 nt, Homo sapiens
  source 1..18: mol_type = mRNA
ccaggaaaaatggcagct

SEQ ID NO: 59 - RNA, 18 nt, Homo sapiens
  source 1..18: mol_type = mRNA
ccaggaaaaatggcagct

SEQ ID NO: 60 - RNA, 18 nt, Homo sapiens
  source 1..18: mol_type = mRNA
caggaaaaatggcagctc

SEQ ID NO: 61 - RNA, 18 nt, Homo sapiens
  source 1..18: mol_type = mRNA
aggaaaaatggcagctct

SEQ ID NO: 62 - RNA, 18 nt, Homo sapiens
  source 1..18: mol_type = mRNA
ggaaaaatggcagctctt

SEQ ID NO: 63 - RNA, 18 nt, Homo sapiens
  source 1..18: mol_type = mRNA
gaaaaatggcagctctta

SEQ ID NO: 64 - RNA, 18 nt, Homo sapiens
  source 1..18: mol_type = mRNA
aaaaatggcagctcttaa

SEQ ID NO: 65 - RNA, 18 nt, Homo sapiens
  source 1..18: mol_type = mRNA
aaaatggcagctcttaaa

SEQ ID NO: 66 - RNA, 18 nt, Homo sapiens
  source 1..18: mol_type = mRNA
aaatggcagctcttaaag

SEQ ID NO: 67 - RNA, 18 nt, Homo sapiens
  source 1..18: mol_type = mRNA
aatggcagctcttaaagg

SEQ ID NO: 68 - RNA, 18 nt, Homo sapiens
  source 1..18: mol_type = mRNA
atggcagctcttaaagga

SEQ ID NO: 69 - DNA, 21 bp, synthetic construct
  source 1..21: mol_type = other DNA
ccaattgggtgcgtaactttg

SEQ ID NO: 70 - DNA, 20 bp, Synthetic construct
  source 1..20: mol_type = other DNA
gttcttctgactggcaacca

SEQ ID NO: 71 - DNA, 24 bp, Synthetic construct
  source 1..24: mol_type = other DNA
  misc_difference 9^10: note = ZEN
acagcccatgttctaccaccttgg

SEQ ID NO: 72 - DNA, 10248 bp, Homo sapiens
  source 1..10248: mol_type = genomic DNA
tgggctcaaacattttctcctttaagagctgccatttttcctggctggtgccataggaatcatctgggtgcctgggcacacccgctgctgctttaaggcttccgccctgatgctgacactgctgctccacgggcccagttctgcatctccaggaaagacaaacagtctccagttttgggcccagctttcctagtctcttcttttccttaccctcagccctgatcttgtgtttgtacggacagtgagctcaccctaggcctggacccaggcccagtttccaaagcaagcagcacacagcgcatgttcacataagcatgggggctggggggacactggggcttactgatctttttctaggggcctccagcccctggcaccacctagaggggaaagtgagtcacccaaaccattgcccctgggcttacgtcgctgtaagctcacactggccctgctgtgccctctttagtcacagacagcgtgtgagctgactctgtccctttaatgcccaggctgagcccagtgcctccttgagtatctgctccatcactggcgacgccacaggtaggtgtgaatggagtagccaggtgagattgtctccaggaagcccacagcaggatccttgatggtaagaggcacatccttagaggagctagggagcagggaggagaagctgagagtgtgatcctgccaaggcccccaacgctgtcttcagcccacttcccagacctcaccattgccctcaccggtttagcacgaccacaacagcagagccatcgggatgcatcagtgccactgcgtccaggtcgttcttctgactggcaaccagccccactctctgggagccctcaggaatgaacttgctgaatgtgggaacagatggtcagagtccctcggggtacctcccatgaaaccctcatctaagaagtcacccacccacggacccaccccataactcctgcagaggctctgccctggctctctaggcctggagccatgctgctgggcactgaccctgcttttctgcatcgcagtccagcctcaggcattggggttttctgttgctacctagtcacttcctgcctccatggtgcaaaaggggatgggtgtgcctcttccgaggttccaccctgaacaccttcctgctccctcgtggtgtagagtgatgtaagccatccgatgtaggagatgataggcctggtatggaatgggggtgcccgccctccactcacctgaagtggccaaggtggtagaacatgggctgtttgtaaaacgtgtccttggtgatgtctacaatgatgggactgtcgacaaagttacgcacccaattgggtcctccttcggggttcagggcaaggttccagtcggtccagccgaccacatggtacaggaggttctagggtaaggacaaaggcaaagagacaaaggcgcaacactgggggtccccagagagtgtaggtaagggtcacatgtgggagaggcagctgtgggtaggtcagccctgtgaggggcacattccttagtagctaaggagttgggggtgtgaagatccaggcatctcaaggggagctgagaagtctgaggcagctgcaagtgcctcagtagttgcaaaaggggcaatgaggtgtgcagacctgtgaaggaaagggaagatagggaatcatggttccccagagttgctcaaaagggcaggctagctggggaaagctggacaggaagggcttctgtcagtctttggtgaaactagtaagaggtctgaggtctgctttgcaggaagggagactggggtggcttaccgtgatgatgctgtggctgtactgcatccctcgatcccaggagcctagccgcacactctgctcccagaacttggagcccacacaggcctctgaggcaaagagcatggtgttggggaacaggcggtgtgtctcccctagggtggctttggctggagccagaaagtccaggtaccaatgtacagcaatgccatgaacatatttagctgcttctgggtctgtcagtacctgcaaaggaagagcaactgatcctggaccttgcacacaggcttctggaacttctagttcctgttgtaggaatcctggagttgggtgacgggaagaatgcaactagagaggtttggggagatttttttttgtttttgagacaggatgtcactctgtcactcgggctggagtacagtggcgcaatcacgactcactgcagccttgacttcctagggtcaactgatcctcccacattagcctcctgagtagctgggactacacgggtgccacacccagctaatttgtgtgtgtatgtgtgtgtatgtatgtgtgtgtgtgtatatatatatacatataaacacatatatatgtatatacacatacatacatgaaccaccacccccagcctagatagttttgttttgttttgtttcgagatggagtctcgctcagcctcccaggctggagtgcagtggcgcgatctcggctcaccgcaaccaccatctcccgggttcaactgattctcccttctcagcctcctgagtagctgggattacagacacccaccatcatgcccagataattttttttttttgtattttagtagacacagggtttcaacacgttggccagggtggtcttaaactcctgacctcaggtgatcacccgcctcggcttcccaaagtgctgggtttgcatgagtgagccacctcgcccagcccctagaaaggtttcaagcgacaactgtgggatccatggcaccctggaggtccaggggaatggtgctctaggaatccatagttgggtagagaaatcgctctaagtttgggagccagtcatttggatgctggatttgaaggtcactggagcaccatggaggtccaggccttaccacctttgcccagtggggcagcagcaagcgttggtcatccagcatgagtaggcggacattgtggtgagtactgttggcgagggtaggacctaggtcacgggcaatgaagtctcgctgatgttcaggggtgaagcccaggcactggaaggggtatccactcaacagcccagcagaaggctcattttcagctgtcactgcccagaactgtaacttgtgctcagcataggcatccaggaacctggcaagagaaaggtcatgaatgatccggccaagaaagtggaccagaccagctgggtgtggtggctcacacctgtaatcccagcactttgggaagccgaggcaggtggatcacttgagttcaggagttcgagaacagcctggcgaaaccccgtctctactaaaaatagaaaaatcagctgggcctggtggcaggcgcctataatcccagctacttggaaggctgaggcaggagaattgcttgaactcaggaggcagaggttacagtgagtgaagatggcgccacagcactccagcctgggtgacagagagagagactccttctcaaaaaaaaaaaaaagaagaaaaataaaaagaaagtgggccagaccgagagaacaggaagcctgatggagtgggcaagattgacaggcccaaggctgaaaggcccagaaggtagaaaggtgagctgaggacaggcagatctggaagtggaactaggttgagggttgggacacagatcagcatggctaaatgggaggccagtcctgatcccacatccttgctgatcccttacttcacaaagtatctggcccaggtctggtggtagatgtctccgggctgtcccttgagtgaccccttcccattcaccgctccattggtcttgagccaagtgggtgatgtccaggggctggcaaggagtgaaacgggacgctgggccaactgcagggctcggtgaatcaggggtatctagagacaaaggtagtgaagagagaagcacccagagttggaacacatactagcccaaccagtgcatccggttcagccattagcctccaccctcccacccccaggacaaaacagcaggggacaaaatgtctgtacaagcagacctaccctacagtttctcaacccccagacatcagggccctcagggcctgaaaaagctagaatgcctaccttgagcttggtatcttcctctgggaggctgaagttgtgcaactggaaatcatcaggggtgtctgcataggtgtaggtgcggatggagaagtcacagctggccatgggtacccggatgatgttatatccgattcctacagaaaaggatgatcaagatatggtagtccgagtcaataggagagtatgggactctgcttatcacttgccagtcctaatagtgtctgagtcagggccaaagggaacttgggctcctgggttggaacctgtggaggctggcacctgggtgaagcgcaggcctttctgagcctgagtccgtagcagttagcagatgataggcggtgaaatcttatttcacagggcattaaaacaggaaccaaatgtcagggatgggcagaagtcagggtccaaagaaagggcaaagaaaagtgtcagtggctcacgcatgtaatcccagcactttgggaggccgatgtgggcagatcacgaggtcaggagttcgcaatcatcctggccaacatagtgaaaccctgtctctactaaaaatacaaaaaattagccgggcgtggtggcaggcacctgtaatcccaggtactcgagaggctgagacaggagaatcacctgaacccgggaggtggaggttgcagtgagctgagattgtgccactgcactccagcctgggtgacagtgcgagactctgtctcaaaaaaaaaaaaagaaaaaagaaaagtgtctgctgggctcggtggctcacacttgtaattccagcacttcgggaggccaaggcaggtatatcatttgaggtcaggagtttaagactagcctggtcaacatgatgaaaccctgtctctactaaaaatacaaaaattagccaggtggtagtggcgcacgcctataattccagctactcaggaggctgaggcaggagaatcactatagcctgggaggcagaggttgctgtgaggggagatcacaccactgcactcctgtctcgccgacagaatgggcagagtgagattctgcctcaaaaaaattttaaaaaaagaaaagaaaaacgaaaagtttcaatggctctatgtcatcttgtccccttcctcctcaccttcttcagagaagtacgatttaagtagcaaattttgggcagggggtgacagggcaaggatgttgagagcagcagcatctgtcatggcccctccaaatcccttcactttctggaacttctgttctggctgcagggtcagtagcaggcctgaggacatccacagggaataagggtatcagtacccagcgggaaactccatggtgatcactgacaccatttacctctaggaggacccagcctggcccagggggtgaggggtgtaatggttacctgtgcccgtgtgattagcctggatgggccccatactcagctccatccgtcgcccactgcgtgtactctcatagcggctgaaggtaccaagggcaggaaaggtcggggggtcaaaggagtcacagtatgtggcattgcagacacacaccaccgagctgtagccgaagcttttagggatgcaggggcgggcacctgggagggagggagtacaagcagagtgaggtctgatgaagacatggagaatggacacatctgctaggagagactgaacacggtttcaaaattcctcaccccttggccgggcgcaggggctcacacctgtaatcctagcactttaggaggccgaggtgggcggatcacctgaggtgaggagtttgagacctgccttgccagcatggtgaaaccccgtctctactaaaaatacaaaaattagctgggcgtagtggtggccgcctgtaatcccagctacctgggaagctgaagcaagagaatcgcttgaatctgggaggcagaggttggaatgagccaaaattgcaccactgcactccagcccaggcaacagagtaagactctgtttcaaaagaaaaaaaacaaaatcctcaccccaaagttggtctcagtcactcaaaaggatttattgagcacctactaaaagtctaccaccagcttactggaaggctaccaaaggactatgaggcagaagggaggctctgtgctacctccccactgccttgactcactcacctgatgcccacgacactgcctgaagtagaagcaatcctgtgaggctgccagccatgatgcttaccctactcaaaggcttgggacattcctgaggacagaatgaggaatgactgaaaagcaagcccctctccacccctcaactactctcctgggcagggcttagctgcctttgggtgcccatggcccggtctcccacattcattaggacaaggcccacagaaacctgggtgcagctctccctgcaacccttctgatgacaactctctgcccaccccaaatcaggatgctccccaccactcttcccacccatttcaacttcgacccctccctccatctgtgccttgctcaaagagccatgatggccctggattcaaagagagtctgtcattcattaaattccagtgccaggattccagaagcactgtgacaatgctgattgggagctctctctcttacctctctggaaggacttgaaaactccatcccctcagggtcattagatgaagagaagaccacaggggttccagagtctctgaaggatagaggatccactaaacaaaaacaaggatgcaggtacctgccttagctataggcactaggttagccctgcaagtaattccggcttcccgatgtggatgggtcatgtgatgactaggagcgtcacatgacacaggaagtgaggcaatcacagccatatttctaaagggcaattggcttcctctcatctgttacagattatatgccctataaaactctggagggcatgtatgggtgacaactttaggaagagcctagaacacagatttgcatggagaatggggtagaggctcctaaatcccagaggatgggaaccacagcaggcacacttcttttttgatagaaatgtcaaaaaggtacaaataaagctgatataatttaaaaaaaaaaacttcactgtgcaaaatactgacactaccaagatgtttaagacagtctttgccctatagaggtgtgtgaggcatggaagtcagacacacagatatttacagataaagacagaaacggtaacaggtgtgcatggagagctctctgagatgaggagggaccattcgtgggtggggaattatccaggatggcttcacagaggaaaaagtgaggggggttattagccagtgaagtgcagggcacaagagggtgggacactggcagtgggatgacaggactggagggagggagtggtcagtgcggctcctctgcagcgtcccttgtttcatcatcagatgcagatggtaataactgttcttccttcctcacaagacagggaggtttgtaaagtcgttcaaaaaccaaagtgttgtacaaagccatatcctcagtggacacaaggaggaagctgtccatggtgtggcctcatgaaccacatcaaatgagatttagcgggagtggcacacacagtcatgacctgactaatcccagctctcagcccatttccttgcctggaaaatggaggcaatgccacaacctcaaagggtggttactgcagtcagtgaggtaagtgcagtgcctgaccacttggtaggcacctgggaactacttgtctcttgtttgtattttttgttgttgttgttttttgagacagagtctcactctgttgcccaggctggagtgcagtggcgtgatcttggctcactgcaacctccacctcctgggttcgagtgattctcctgcctcagcctcccaagtagctgggattacaggtgccctccaacatgctcagctaatttttttatttttagtagagatggggtttcactatgttggccaggctggtctgaaactctggacctcaggtgatctgctgccttggtatccgaaagtgctgggattacaggcatgagccactgcgctggaccccagccatcctttttgttctttaaatgatctcagtgaagtctttctagatacactccaagcaaaattgatcatttcttcttctaggttcctccagtaatttttttttttttggttttgagacagagtcttgccctgttgcccaggctggagtgcagtgtgatctcggctcaagcaattgccctgcctcagcctcctgagtagctgggattacaggagcccaccaccatgcccagctaatttttgtatttttagtagagatggggtttcaccatgttggccaggctggtctcgaacccctgacctcaggtgatccaccagcctcagcctcccaaggtgttgggattatagcaatgagccaccacacccggtccctccagtaattaagtacagtctcagtgagacagcaagtttggaatcctggctacaccatttactggctgtttgactttcaataaatcaatcactctaagcctctgtttcatctataaaaggggagtgataactcctacctcacagaattgttgtgaggtttgagtgtgataatgtgtctctagtacactgcttgacactaaacattgcaacatgtcaggcctctgttcctggagttccttgaaggaatgtcttatgcattctaagtatcctcgtagatagcctggcacaggggtagctgtcaagttgtagaattgaactagttgcttcactatgtcagtagccaccccttccagacttctcacttttcaaggaaacattgcacaggatttgttctgggtagggcaggtaatatctagtaccttacttccctcaagttcattcatctcacagatatttcctgagcacattctacatttgcctctcctgctctattgagtttagaagtccaaacatctccttccacttcccctctgcatagtgagcctcttcttttttggccaggtactgagcaattttttgtttgtttgtttgagacagggtctccgtatgttgccctggatggagtgtagtagcttgatttcggctcagtgcaacctctgcctccagaataagcaatcctcccacctcagcctccagaatagctgggactataggcgcacaccaccacacctggctaatttctgtattttttgtagagacggggtcttactgtgttgcgcaggttggtctcaaactcctaaactcaaaggatcctcccgccttggctttgcaaagtgctgggattacagggtgaggcactgcgcccagtccgcttgttttttgttttgtttttttttttgagacggagtatctctgtcacccaggctggagtgcagtggcacgatctcggttcactgcaacctccgcctcctaggttcaagcggagggtagggaccagtccatcctggcacccatctgcagctccagtgcgaatcccaaccccgacgctcgtcgccgggctccgtgaatgtttgtcacatgtctgaagaacgtatgaattacataaccttcttcccactccacccctcaaaaagcaagtggatataaagacttgaagattttataatctcttcttcattagtaaaatctgaccatccttaccgttaaaaataataatgatggttggccgggcgcggtggctcacgcctataatcacagctgtttggcaggccgaggcgggcggatcacgaggtcaggaattggagaccagcctgaccaacatggtgaaaccccgtctctactaaaagatacaaaaaattagccgtgcgtggtggcgggtgcctgtaatcccacgtacttgggaggctgaggtcggagaatcgcctgaacccgggaggcggaggttgcagtgagccaagaaggcgccattacactccagcctgggcgacagggcgagactccctcaaaaaataataataataacaataataataataatggtgaaaaaggttaagtgcgaacgcagggaggggacagctaagatccaaaggtcgaaatattcattacgcctaccgtcggcgaagagaaacagcagccccaaccggaggcaaaacgaaatcccaccgcagcctgcaaaggcgcctgggcgggactggagactggggccccgcgcagtaagactctgaaggcaggatgcagcccgaccacccgcagccgcgagaaaagcagccctggggagtcggggcgggacctggattggaaaagag

SEQ ID NO: 73 - RNA, 2291 nt, Homo sapiens
  source 1..2291: mol_type = mRNA
ggaattacttgcagggctaacctagtgcctatagctaaggcaggtacctgcatccttgtttttgtttagtggatcctctatccttcagagactctggaacccctgtggtcttctcttcatctaatgaccctgaggggatggagttttcaagtccttccagagaggaatgtcccaagcctttgagtagggtaagcatcatggctggcagcctcacaggattgcttctacttcaggcagtgtcgtgggcatcaggtgcccgcccctgcatccctaaaagcttcggctacagctcggtggtgtgtgtctgcaatgccacatactgtgactcctttgaccccccgacctttcctgcccttggtaccttcagccgctatgagagtacacgcagtgggcgacggatggagctgagtatggggcccatccaggctaatcacacgggcacaggcctgctactgaccctgcagccagaacagaagttccagaaagtgaagggatttggaggggccatgacagatgctgctgctctcaacatccttgccctgtcaccccctgcccaaaatttgctacttaaatcgtacttctctgaagaaggaatcggatataacatcatccgggtacccatggccagctgtgacttctccatccgcacctacacctatgcagacacccctgatgatttccagttgcacaacttcagcctcccagaggaagataccaagctcaagatacccctgattcaccgagccctgcagttggcccagcgtcccgtttcactccttgccagcccctggacatcacccacttggctcaagaccaatggagcggtgaatgggaaggggtcactcaagggacagcccggagacatctaccaccagacctgggccagatactttgtgaagttcctggatgcctatgctgagcacaagttacagttctgggcagtgacagctgaaaatgagccttctgctgggctgttgagtggataccccttccagtgcctgggcttcacccctgaacatcagcgagacttcattgcccgtgacctaggtcctaccctcgccaacagtactcaccacaatgtccgcctactcatgctggatgaccaacgcttgctgctgccccactgggcaaaggtggtactgacagacccagaagcagctaaatatgttcatggcattgctgtacattggtacctggactttctggctccagccaaagccaccctaggggagacacaccgcctgttccccaacaccatgctctttgcctcagaggcctgtgtgggctccaagttctgggagcagagtgtgcggctaggctcctgggatcgagggatgcagtacagccacagcatcatcacgaacctcctgtaccatgtggtcggctggaccgactggaaccttgccctgaaccccgaaggaggacccaattgggtgcgtaactttgtcgacagtcccatcattgtagacatcaccaaggacacgttttacaaacagcccatgttctaccaccttggccacttcagcaagttcattcctgagggctcccagagagtggggctggttgccagtcagaagaacgacctggacgcagtggcactgatgcatcccgatggctctgctgttgtggtcgtgctaaaccgctcctctaaggatgtgcctcttaccatcaaggatcctgctgtgggcttcctggagacaatctcacctggctactccattcacacctacctgtggcgtcgccagtgatggagcagatactcaaggaggcactgggctcagcctgggcattaaagggacagagtcagctcacacgctgtctgtgactaaagagggcacagcagggccagtgtgagcttacagcgacgtaagcccaggggcaatggtttgggtgactcactttcccctctaggtggtgccaggggctggaggcccctagaaaaagatcagtaagccccagtgtccccccagcccccatgcttatgtgaacatgcgctgtgtgctgcttgctttggaaactgggcctgggtccaggcctagggtgagctcactgtccgtacaaacacaagatcagggctgagggtaaggaaaagaagagactaggaaagctgggcccaaaactggagactgtttgtctttcctggagatgcagaactgggcccgtggagcagcagtgtcagcatcagggcggaagccttaaagcagcagcgggtgtgcccaggcacccagatgattcctatggcaccagccaggaaaaatggcagctcttaaaggagaaaatgtttgagccca

SEQ ID NO: 74 - RNA, 22 nt, Homo sapiens
  source 1..22: mol_type = other RNA
aagctgccagttgaagaactgt

(19) United States
(12) Patent Application Publication
(10) Pub. No.: US 2026/0242796 A1
(43) Pub. Date: Aug. 20, 2026

(54) OLIGONUCLEOTIDES CAPABLE OF UPREGULATING GLUCOCEREBROSIDASE EXPRESSION

(71) Applicant: Hoffmann-La Roche Inc., Little Falls, NJ (US)

(72) Inventor: Johannes BRAUN, Grenzach-Wyhlen (DE)

(73) Assignee: Hoffmann-La Roche Inc., Little Falls, NJ (US)

(21) Appl. No.: 19/368,918

(22) Filed: Oct. 24, 2025

Related U.S. Application Data
(63) Continuation of application No. PCT/EP2024/061871, filed on Apr. 30, 2024.

(30) Foreign Application Priority Data
May 4, 2023 (EP) 23171586.3

(51) Int. Cl. C12N 15/113; A61K 31/7105; A61K 31/712; A61K 31/7125; A61K 31/713; A61P 25/28

(52) U.S. Cl. CPC C12N 15/1137; A61K 31/7105; A61K 31/712; A61K 31/7125; A61K 31/713; A61P 25/28; C12N 2310/113; C12N 2310/14; C12N 2310/315; C12N 2310/321; C12N 2310/3231; C12N 2310/345; C12N 2310/3519; C12Y 302/01045

(57) ABSTRACT

Provided are oligonucleotides that increase the expression of glucocerebrosidase (GBA) in cells; conjugates, salts and pharmaceutical compositions thereof; and methods for treatment of diseases associated with reduced expression of GBA, including Gaucher's disease and/or Parkinson's disease. The oligonucleotides may comprise a contiguous sequence complementary to contiguous bases in the 3′ untranslated region (UTR) of the GBA mRNA transcript.

### CROSS REFERENCE TO RELATED APPLICATIONS

This application is a continuation of International Application No. PCT/EP2024/061871 having an International Filing Date of 30 Apr. 2024, claiming priority to application number EP 23171586.3 filed 4 May 2023, each of which is incorporated herein by reference in its entirety.

### SEQUENCE LISTING

This application contains a Sequence Listing which has been submitted electronically in XML format and is hereby incorporated by reference in its entirety. Said XML copy, created on Sep. 30, 2025, is named “P37795-US-1_Sequence-Listing.xml” and is 131,262 bytes in size.

### FIELD OF INVENTION

The present invention relates to oligonucleotides that can increase the expression of glucocerebrosidase (GBA) in cells; conjugates, salts, and pharmaceutical compositions thereof; and their use in methods for treatment of diseases and disorders associated with reduced expression of GBA, including Gaucher's disease and Parkinson's disease.

### BACKGROUND

Glucocerebrosidase (GBA) is a lysosomal enzyme that catalyses the hydrolysis of glucocerebroside (also known as glucosylceramide). Glucocerebroside is a normal component of cell membranes, particularly those of red and white blood cells.

Homozygous mutations in the gene encoding GBA cause Gaucher's disease. During routine cell turnover, macrophages engulf and degrade cell debris. Insufficient GBA activity results in the accumulation of glucocerebroside in the lysosomes of macrophages. Affected macrophages, known as ‘Gaucher cells’, build up in areas such as the spleen, liver and bone marrow.

Gaucher's disease is characterised by bruising, fatigue, anaemia, low blood platelet count and enlargement of the liver and spleen. The phenotype is variable, however three clinical forms have been identified: type 1 is the most common and typically causes no neurological damage, whereas types 2 and 3 are characterised by neurological impairment.

The condition is inherited in an autosomal recessive pattern. Over 300 variants of the GBA gene have been associated with the disease. Although genetics alone does not determine disease severity, certain mutations are known to cause more severe symptoms. For example, patients with two copies of the L444P mutation usually exhibit neuronopathic forms of the disease, whereas patients with one or two copies of the N370S allele are typically classified as type 1 (Scott et al., 2000, Genet. Med., 2, 65).

Mutations in the GBA gene have also been linked to Parkinson's disease and dementia with Lewy bodies (Riboldi and Di Fonzo, 2019, Cells, 8, 364). Parkinson's disease is a neurodegenerative disorder of the central nervous system characterised by a wide range of motor and non-motor symptoms. Motor symptoms include bradykinesia (slowness of movement), rigidity, and postural instability. Non-motor symptoms, which may precede motor symptoms by many years, include olfactory loss, rapid eye movement sleep behaviour disorders, dysautonomia, and depression.

Heterozygous mutations of the GBA gene occur in around 8 to 12% of patients with Parkinson's disease. As with Gaucher's disease, mutation severity can influence the disease phenotype. For example, the risk for dementia in patients carrying “severe” mutations (such as L444P) is 2- to 3-fold higher than in those carrying “mild” mutations (such as N370S). E326K is the most prevalent GBA mutation in Parkinson's disease, and patients bearing this mutation show a faster progression of motor symptoms (Avenali et al., 2020, Front. Aging Neurosci.).

Current treatments for diseases associated with reduced GBA expression include enzyme replacement therapy (ERT) and substrate reduction therapy (SRT). ERT involves the intravenous administration of recombinant GBA. While most patients respond well to treatment, there is a risk of developing an immune response. Furthermore, GBA is not able to cross the blood-brain barrier and therefore ERT is considered ineffective for patients with Parkinson's disease or neuronopathic forms of Gaucher's disease. SRT provides an alternative (or supplementary) treatment for patients who cannot tolerate ERT, or for whom intravenous administration is problematic. SRT works to reduce the build-up of glucocerebroside in the lysosome by inhibiting enzymes in the glucocerebroside synthesis pathway. This therapy has a higher incidence of adverse effects than ERT, and long-term reduction of glucocerebroside can affect several different cell functions. Both ERT and SRT are costly and must be continued for life.

There is a need for therapeutic agents that can increase or restore the expression of GBA.

### SUMMARY OF INVENTION

The present invention relates to antisense oligonucleotides that can increase the expression of GBA, particularly antisense oligonucleotides comprising a contiguous sequence complementary to contiguous bases in the 3′ untranslated region (UTR) of the GBA mRNA transcript.

In a first aspect, the invention provides an antisense oligonucleotide of 8 to 40 nucleotides in length, which comprises a contiguous nucleotide sequence complementary to at least 6 contiguous bases in the 3′ untranslated region (UTR) of an RNA sequence encoding glucocerebrosidase (GBA).

In some embodiments, the antisense oligonucleotide is capable of increasing GBA expression.

In some embodiments, the antisense oligonucleotide is capable of decreasing downregulation of GBA expression mediated by a microRNA (miR) in a target cell, wherein the miR is selected from miR-22-3p and a variant thereof comprising a seed region fully complementary to GGCAGCT.

In some embodiments, the antisense oligonucleotide is capable of inhibiting the binding of the RNA sequence to a miR selected from miR-22-3p and a variant thereof comprising a seed region fully complementary to GGCAGCT.

In some embodiments, the contiguous nucleotide sequence is 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40 nucleotides in length.

In some embodiments, the contiguous nucleotide sequence is 16, 18 or 20 nucleotides in length.

In some embodiments, the contiguous nucleotide sequence is the same length as the antisense oligonucleotide.

In some embodiments, the contiguous nucleotide sequence is at least 75%, at least 80%, at least 85%, at least 90%, at least 95%, or fully, complementary to contiguous bases located downstream of the stop codon TAG at positions 1746 to 1748 in SEQ ID NO:73.

In some embodiments, the contiguous nucleotide sequence is at least 80%, at least 85%, at least 90%, at least 95%, or fully, complementary to a miR-22-3p binding site located downstream of the stop codon TAG at positions 1746 to 1748 in SEQ ID NO:73.

In some embodiments, the contiguous nucleotide sequence is at least 75%, at least 80%, at least 85%, at least 90%, at least 95%, or fully, complementary to contiguous bases located in the segment at positions 2227 to 2274 in SEQ ID NO:73.

In some embodiments, the RNA sequence encoding GBA is an mRNA sequence comprising a 3′UTR sequence comprising the contiguous bases of positions 2227 to 2274 of SEQ ID NO:73.

In some embodiments, the RNA sequence encoding GBA is an mRNA sequence comprising the sequence of SEQ ID NO:73 or an allelic variant thereof.

In some embodiments, the contiguous nucleotide sequence is complementary to a target nucleic acid sequence selected from the group consisting of SEQ ID NO:35, SEQ ID NO:36, SEQ ID NO:37, SEQ ID NO:38, SEQ ID NO:39, SEQ ID NO:40, SEQ ID NO:41, SEQ ID NO:42, SEQ ID NO:43, SEQ ID NO:44, SEQ ID NO:45, SEQ ID NO:46, SEQ ID NO:47, SEQ ID NO:48, SEQ ID NO:49, SEQ ID NO:50, SEQ ID NO:51, SEQ ID NO:52, SEQ ID NO:53, SEQ ID NO:54, SEQ ID NO:55, SEQ ID NO:56, SEQ ID NO:57, SEQ ID NO:58, SEQ ID NO:59, SEQ ID NO:60, SEQ ID NO:61, SEQ ID NO:62, SEQ ID NO:63, SEQ ID NO:64, SEQ ID NO:65, SEQ ID NO:66, SEQ ID NO:67, SEQ ID NO:68, or to a fragment of at least 10 nucleotides of any thereof.

In some embodiments, the antisense oligonucleotide is a single-stranded antisense oligonucleotide.

In some embodiments, the antisense oligonucleotide is a double-stranded oligonucleotide.

In some embodiments, the contiguous nucleotide sequence has or comprises a nucleobase sequence selected from the group consisting of SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO:11, SEQ ID NO:12, SEQ ID NO:13, SEQ ID NO:14, SEQ ID NO:15, SEQ ID NO:16, SEQ ID NO:17, SEQ ID NO:18, SEQ ID NO:19, SEQ ID NO:20, SEQ ID NO:21, SEQ ID NO:22, SEQ ID NO:23, SEQ ID NO:24, SEQ ID NO:25, SEQ ID NO:26, SEQ ID NO:27, SEQ ID NO:28, SEQ ID NO:29, SEQ ID NO:30, SEQ ID NO:31, SEQ ID NO:32, SEQ ID NO:33, and SEQ ID NO:34, or a fragment of at least 10 nucleotides of any thereof.

In some embodiments, the contiguous nucleotide sequence has or comprises a nucleobase sequence selected from the group consisting of SEQ ID NO:2, SEQ ID NO:3, SEQ ID NO:4, SEQ ID NO:5, SEQ ID NO:6, SEQ ID NO:7, SEQ ID NO:8, SEQ ID NO:10, SEQ ID NO:11, SEQ ID NO:12, SEQ ID NO:16, SEQ ID NO:19, SEQ ID NO:20, SEQ ID NO:21, SEQ ID NO:22, SEQ ID NO:23, SEQ ID NO:24, SEQ ID NO:25, SEQ ID NO:26, and SEQ ID NO:27.

In some embodiments, the antisense oligonucleotide comprises one or more modified nucleoside(s).

In some embodiments, the antisense oligonucleotide comprises one or more modified nucleoside(s) independently selected from the group consisting of 2′-O-alkyl-RNA, 2′-O-methyl-RNA, 2′-alkoxy-RNA, 2′-O-methoxyethyl-RNA, 2′-amino-DNA, 2′-fluoro-DNA, arabino nucleic acid (ANA), 2′-fluoro-ANA, morpholino, and locked nucleic acid (LNA) nucleosides.

In some embodiments, the antisense oligonucleotide comprises one or more 2′-MOE RNA nucleosides.

In some embodiments, the antisense oligonucleotide comprises one or more LNA nucleosides, such as beta-D-oxy-LNA nucleosides.

In some embodiments, the antisense oligonucleotide comprises one or more 2′-O-methyl RNA nucleosides.

In some embodiments, the antisense oligonucleotide is a mixmer or a totalmer, optionally wherein the mixmer does not comprise any DNA or RNA nucleosides.

In some embodiments, the antisense oligonucleotide is a mixmer of 2′-MOE and LNA nucleosides or a totalmer of 2′-MOE nucleosides.

In some embodiments, the antisense oligonucleotide comprises at least one modified internucleoside linkage.

In some embodiments, the antisense oligonucleotide comprises one or more phosphorothioate internucleoside linkages.

In some embodiments, all internucleoside linkages in the antisense oligonucleotide are phosphorothioate internucleoside linkages.

In some embodiments, the antisense oligonucleotide is capable of increasing the expression of GBA by at least about 5%, at least about 10%, at least about 15%, at least about 20%, at least about 30%, at least about 40%, at least about 50%, or more than 50%, optionally compared to a control.

In some embodiments, the antisense oligonucleotide is covalently attached to at least one conjugate moiety.

In some embodiments, the antisense oligonucleotide is in the form of a pharmaceutically acceptable salt.

In some embodiments, the pharmaceutically acceptable salt is a sodium salt or a potassium salt

In some embodiments, the antisense oligonucleotide is encapsulated in a lipid-based delivery vehicle, covalently linked to or encapsulated in a dendrimer, or conjugated to an aptamer.

In a second aspect, the invention provides a pharmaceutical composition comprising the antisense oligonucleotide according to the first aspect, and a pharmaceutically acceptable diluent, solvent, carrier, salt and/or adjuvant.

In some embodiments, the pharmaceutical composition comprises an aqueous diluent or solvent, such as phosphate buffered saline.

In a third aspect, the invention provides the antisense oligonucleotide according to the first aspects or the pharmaceutical composition according to the second aspect for use as a medicament.

In a fourth aspect, the invention provides an in vitro or in vivo method for increasing or restoring GBA expression in a target cell, the method comprising administering an effective amount of the antisense oligonucleotide according to the first aspect or the pharmaceutical composition according to the second aspect to the target cell.

In some embodiments, the cell is a mammalian cell, such as a human cell.

In some embodiments, the expression of GBA is increased by at least about 5%, at least about 10%, at least about 15%, at least about 20%, at least about 30%, at least about 40%, at least about 50%, or more than 50%, compared to a control.

In some embodiments, the control is a target cell to which the antisense oligonucleotide is not administered.

In a fifth aspect, the invention provides a method for treating or preventing a disease or disorder, comprising administering a therapeutically or prophylactically effective amount of the antisense oligonucleotide according to the first aspect, or the pharmaceutical composition according to the second aspect to a subject suffering from or susceptible to a disease or disorder.

In a sixth aspect, the invention provides the antisense oligonucleotide according to the first aspect or the pharmaceutical composition according to the second aspect for use in treating or preventing a disease or disorder.

In a seventh aspect, the invention provides a use of the antisense oligonucleotide according to the first aspect or the pharmaceutical composition according to the second aspect for the preparation of a medicament for treatment or prevention of a disease or disorder in a subject.

In some embodiments of the method according to the fifth aspect, the antisense oligonucleotide or pharmaceutical composition for use according to the sixth aspect, or the use according to the seventh aspect, the disease or disorder is associated with reduced expression of GBA.

In some embodiments, the disease is selected from the group consisting of Gaucher's disease, Parkinson's Disease, dementia, dementia with Lewy bodies (DLE) and rapid eye movements (REM) sleep behaviour disorders.

In some embodiments, the disease is Parkinson's disease.

In some embodiments, the disease is Gaucher's disease.

These and other aspects and embodiments of the invention are described in further detail below.

### BRIEF DESCRIPTION OF FIGURES

FIG. 1 shows GBA mRNA expression levels in H4 neuroglioma cells at 48 hours post-transfection relative to a mock transfection control. Grey and black bars represent antisense oligonucleotide concentrations of 5 nM and 25 nM, respectively. Antisense oligonucleotides are here referred to by SEQ ID NO, which has the same number as their corresponding COMP ID NO.

### DETAILED DESCRIPTION OF THE INVENTION

The inventors have identified that the expression level of GBA protein products can be effectively increased by targeting the GBA mRNA transcript with antisense oligonucleotides. In particular, they have surprisingly determined that targeting the 3′ untranslated region (3′UTR) of the GBA mRNA transcript can be effective.

Described herein are target sites present on the human GBA nucleic acid target, such as a GBA mRNA sequence, which can be targeted by antisense oligonucleotides of the invention.

Without wishing to be bound by theory, it is considered that the antisense oligonucleotides interfere with microRNA (miR)-mediated degradation of the GBA mRNA transcript, thereby increasing the expression of GBA protein. Particularly, the antisense oligonucleotides are considered to interfere with degradation of the GBA mRNA transcript mediated by miR-22-3p or a variant thereof.

MiR-22-3p (SEQ ID NO:74) is a non-coding RNA which may act as a single-stranded guide sequence for the miRNA-induced silencing complex (miRISC) to induce GBA mRNA degradation and translational repression. Contemplated variants of miR-22-3p include those which comprise a seed region fully complementary to GGCAGCT as well as those comprising a seed region which is fully complementary to GGCAGCT except for one or two mismatches, such as one mismatch. For example, variants of MiR-22-3p may have a % sequence identity of at least 80%, such as at least 85%, such as at least 90%, such as at least 95%, to SEQ ID NO:74 and comprise a seed region fully complementary to GGCAGCT, or with one or two mismatches. Preferably, variants of MiR-22-3p comprise a seed region fully complementary to GGCAGCT.

### Increasing Expression of GBA

The oligonucleotides of the invention are capable of increasing the expression of GBA.

Increased GBA expression is desirable to treat a range of disorders which are characterised by, or caused by, reduced expression of GBA. These include Gaucher's disease, Parkinson's disease dementia, dementia with Lewy bodies (DLB) and rapid eye movements (REM) sleep behaviour disorders.

Unless otherwise indicated or contradicted by context, the terms “increasing the expression of GBA,” “upregulating GBA”, “enhancing the GBA level,” “restoring GBA expression” and the like as used herein are to be understood to refer or relate to increasing GBA mRNA transcripts, increasing GBA protein or increasing both GBA mRNA and GBA protein, typically in a cell.

Advantageously, the increase in GBA expression effected by an oligonucleotide can be identified in a cell exposed to the oligonucleotide as compared to a control. The control is typically the GBA expression level in a cell that has not been exposed to the oligonucleotide. For example, the control cell can be a cell which has been treated with a non-targeting oligonucleotide or a cell which has been exposed to a mock transfection, e.g., in which it was only treated with PBS. Alternatively, the control may be a control GBA value referring to the level of GBA mRNA and/or GBA protein in a cell before exposure to the oligonucleotide. Also, when evaluating the ability of an oligonucleotide to restore GBA expression, the cell in which the increase in GBA expression is tested can be a cell which has a lower-than-normal GBA expression and the control can be a cell with normal expression of GBA or a control value reflecting normal GBA level.

In certain embodiments, oligonucleotides capable of increasing the expression of GBA may increase GBA mRNA levels by at least about 5% compared to a control. More preferably the oligonucleotides capable of increasing the expression of GBA may increase GBA mRNA levels by at least about 10%, such as at least about 15%, at least about 20%, at least about 25%, at least about 30%, at least about 35%, at least about 40%, at least about 45%, at least about 50%, at least about 60%, at least about 70%, at least about 80%, at least about 90%, at least about 100%, at least about 200%, at least about 300%, at least about 400%, at least about 500%, at least about 600%, or more compared to a control.

In certain embodiments, oligonucleotides capable of increasing the expression of GBA may increase GBA protein levels by at least about 5% compared to a control. More preferably the oligonucleotides capable of increasing the expression of GBA of the present invention may increase GBA protein levels by at least about 10%, such as at least about 15%, at least about 20%, at least about 25%, at least about 30%, at least about 35%, at least about 40%, at least about 45%, at least about 50%, at least about 60%, at least about 70%, at least about 80%, at least about 90%, at least about 100%, at least about 200%, at least about 300%, at least about 400%, at least about 500%, at least about 600%, or more compared to a control.

In certain embodiments the oligonucleotides capable of increasing the expression of GBA of the present invention may increase GBA mRNA and protein levels by at least about 5% compared to a control. More preferably the oligonucleotides capable of increasing the expression of GBA of the present invention may increase GBA mRNA and protein levels by at least about 10%, such as at least about 15%, at least about 20%, at least about 25%, at least about 30%, at least about 35%, at least about 40%, at least about 45%, at least about 50%, at least about 60%, at least about 70%, at least about 80%, at least about 90%, at least about 100%, at least about 200%, at least about 300%, at least about 400%, at least about 500%, at least about 600%, or more compared to a control.

In certain embodiments, oligonucleotides capable of increasing the expression of GBA may increase GBA mRNA levels by at least about 5% compared to a control. More preferably the oligonucleotides capable of increasing the expression of GBA may increase GBA mRNA levels by at least about 10%, such as at least about 15%, at least about 20%, at least about 25%, at least about 30%, at least about 35%, at least about 40%, at least about 45%, at least about 50%, at least about 60%, at least about 70%, at least about 80%, at least about 90%, at least about 100%, at least about 200%, at least about 300%, at least about 400%, at least about 500%, at least about 600%, or more compared to a control.

In certain embodiments, oligonucleotides capable of increasing the expression of GBA may increase GBA protein levels by at least about 5% compared to a control. More preferably the oligonucleotides capable of increasing the expression of GBA of the present invention may increase GBA protein levels by at least about 10%, such as at least about 15%, at least about 20%, at least about 25%, at least about 30%, at least about 35%, at least about 40%, at least about 45%, at least about 50%, at least about 60%, at least about 70%, at least about 80%, at least about 90%, at least about 100%, at least about 200%, at least about 300%, at least about 400%, at least about 500%, at least about 600%, or more compared to a control.

In certain embodiments the oligonucleotides capable of increasing the expression of GBA may increase GBA mRNA and protein levels by at least about 5% compared to a control. More preferably the oligonucleotides capable of increasing the expression of GBA of the present invention may increase GBA mRNA and protein levels by at least about 10%, such as at least about 15%, at least about 20%, at least about 25%, at least about 30%, at least about 35%, at least about 40%, at least about 45%, at least about 50%, at least about 60%, at least about 70%, at least about 80%, at least about 90%, at least about 100%, at least about 200%, at least about 300%, at least about 400%, at least about 500%, at least about 600%, or more compared to a control.

In certain embodiments, oligonucleotides capable of restoring the expression of GBA may restore GBA mRNA levels to at least about 5% compared to a control. More preferably the oligonucleotides capable of restoring the expression of GBA may restore GBA mRNA levels to at least about 10%, such as at least about 15%, such as at least about 20%, at least about 25%, at least about 30%, at least about 35%, at least about 40%, at least about 45%, at least about 50%, at least about 60%, at least about 70%, at least about 80%, at least about 90%, or more compared to a control.

In certain embodiments, oligonucleotides capable of restoring the expression of GBA may restore GBA protein levels to at least about 5% compared to a control. More preferably the oligonucleotides capable of restoring the expression of GBA of the present invention may restore GBA protein levels to at least about 10%, such as at least about 15%, such as at least about 20%, at least about 25%, at least about 30%, at least about 35%, at least about 40%, at least about 45%, at least about 50%, at least about 60%, at least about 70%, at least about 80%, at least about 90%, or more compared to a control.

In certain embodiments the oligonucleotides capable of restoring the expression of GBA of the present invention may restore GBA mRNA and protein levels to at least about 5% compared to a control. More preferably the oligonucleotides capable of restoring the expression of GBA of the present invention may restore GBA mRNA and protein levels to at least about 10%, such as at least about 15%, such as at least about 20%, at least about 25%, at least about 30%, at least about 35%, at least about 40%, at least about 45%, at least about 50%, at least about 60%, at least about 70%, at least about 80%, at least about 90%, or more compared to a control.

Without wishing to be bound by theory, the increase effected by the oligonucleotides is believed to relate to their ability to reduce, remove, prevent, lessen, lower or terminate the degradation or translational repression of a GBA mRNA transcript, e.g., by their binding to the 3′-UTR region of the GBA mRNA transcript, thereby reducing, blocking or preventing the binding of the miRNA-induced silencing complex (miRISC) mediated by MiR-22-3p, or variants thereof, to the 3′-UTR region of the GBA mRNA transcript. The increase can also be viewed as the oligonucleotides' ability to restore expression of GBA to normal levels, e.g., by reducing or removing degradation of the GBA mRNA transcript or increasing its translational output.

Consequently, the oligonucleotides of the invention may also or alternatively be capable of decreasing downregulation of GBA expression mediated by a microRNA (miR) in a target cell, wherein the miR is selected from MiR-22-3p and a variant thereof. Preferably, the MiR-22-3p variant comprises a seed region fully complementary to at least 6 contiguous nucleotides of GGCAGCT, such as fully complementary to GGCAGCT.

Downregulation of GBA expression as used herein is to be understood to refer or relate to downregulation of GBA mRNA levels, downregulation of GBA protein levels or downregulation of both GBA mRNA and GBA protein levels, typically in a cell, and advantageously in comparison to a control. The control is typically the degree of downregulation of GBA levels in a cell that has not been exposed to the oligonucleotide. For example, the control cell can be a cell which has been treated with a non-targeting oligonucleotide or a cell which has been exposed to a mock transfection, for example, in which it was only treated with PBS. Alternatively, the control may be a control value referring to the degree of downregulation of GBA mRNA and/or GBA protein in a cell before exposure to the oligonucleotide. A decrease in downregulation of GBA expression typically leads to an increase in GBA levels, as described herein.

Preferably, the oligonucleotides of the present invention may decrease downregulation of GBA expression mediated by miR-22-3p, or a variant thereof, by at least about 5%, such as at least about 10%, at least about 15%, at least about 20%, at least about 25%, at least about 30%, at least about 35%, at least about 40%, at least about 45%, at least about 50%, at least about 60%, at least about 70%, at least about 80%, at least about 90%, at least about 95%, or about 100%, as compared to a control.

A suitable assay for evaluating the effect of oligonucleotides on GBA mRNA expression is described in Example 1. Other suitable assays for evaluating GBA mRNA and/or GBA protein levels or miR-22-3p-mediated downregulation of GBA expression (see, e.g., Straniero et al., Sci Rep. 2017 Oct. 5; 7(1):12702), are known in the art.

Particularly contemplated oligonucleotides of the invention include antisense oligonucleotides of 8 to 40 nucleotides in length, which comprise a contiguous nucleotide sequence complementary to at least 6, such as at least 7, such as at least 8 contiguous bases in the 3′ untranslated region (UTR) of an RNA sequence encoding GBA. The RNA sequence encoding GBA is preferably an mRNA sequence comprising the sequence of SEQ ID NO:73 or an allelic variant thereof.

The oligonucleotide of the invention may also or alternatively be capable of inhibiting the binding of a miR selected from miR-22-3p and a variant thereof, to an RNA sequence encoding GBA. Preferably, the variant comprises a seed region fully complementary to GGCAGCT.

Unless otherwise indicated or contradicted by context, the terms “inhibiting”, “blocking” or “decreasing” the binding of an RNA sequence encoding GBA to a miR, such as miR-22-3p or a variant thereof, are to be understood to refer or relate to the ability of the oligonucleotide to reduce the amount of miR binding to the RNA sequence, preferably in comparison to a control. A suitable control may be the amount of miR binding to the RNA sequence in the absence of the oligonucleotide, or the level of miR binding to the RNA sequence in the presence of an irrelevant control oligonucleotide.

Preferably, the oligonucleotides of the present invention may inhibit the binding of an RNA sequence encoding GBA to a miR selected from miR-22-3p and a variant thereof by at least about 5%, such as at least about 10%, at least about 15%, at least about 20%, at least about 25%, at least about 30%, at least about 35%, at least about 40%, at least about 45%, at least about 50%, at least about 60%, at least about 70%, at least about 80%, at least about 90%, at least about 95%, or about 100%, as compared to a control.

The invention also relates to blockmirs. As used herein, a “blockmir” is an oligonucleotide comprising a contiguous nucleotide sequence that is complementary to an mRNA sequence, typically an untranslated region of an mRNA, that is targeted by a miRNA. A blockmir may therefore be capable of at least partially inhibiting or blocking the miRNA from binding to the same site.

Accordingly, in some embodiments, the oligonucleotides of the invention are blockmirs, particularly blockmirs complementary to an mRNA sequence targeted by miRNA-22-3p or a variant thereof. Preferably, the untranslated region of the mRNA that is targeted by the miRNA-22-3p or variant thereof is the 3′ UTR of the mRNA sequence encoding GBA.

Particularly contemplated blockmirs of the invention include oligonucleotides of 8 to 40 nucleotides in length, which comprise a contiguous nucleotide sequence complementary to at least 6, such as at least 7, such as at least 8 contiguous bases in the 3′ untranslated region (UTR) of an RNA sequence encoding GBA. The RNA sequence encoding GBA is preferably an mRNA sequence comprising the sequence of SEQ ID NO:73 or an allelic variant thereof.

Target sites present in the 3′ UTR of the human GBA mRNA sequence which can suitably be targeted by the oligonucleotides, e.g., blockmirs, of the invention are described herein.

The invention also relates to GBA agonists. As used herein, the term “GBA agonist” refers to a compound, in this case an oligonucleotide or conjugate thereof, which is capable of increasing GBA, i.e., GBA mRNA transcripts, GBA protein, or both GBA mRNA transcripts and GBA protein, in a cell. Typically, the cell is a cell which is capable of expressing some GBA mRNA transcripts and/or GBA protein. As described herein, Enhanced GBA expression is desirable to treat, for example, Gaucher's disease and/or Parkinson's disease.

Advantageously, GBA agonist activity can be identified in a cell exposed to the GBA agonist as compared to a control. The control is typically the GBA in a cell that has not been exposed to the oligonucleotide. For example, the control cell can be a cell which has been treated with a non-targeting oligonucleotide or a cell which has been exposed to a mock transfection, for example, in which it was only treated with PBS. Alternatively, the control may be a control GBA value referring to the GBA mRNA and/or GBA protein in a cell before exposure to the GBA agonist. Also, when evaluating GBA agonist activity, the cell in which the increase in GBA expression is tested can be a cell which has a lower-than-normal GBA expression and the control can be a cell with normal expression of GBA or a control value reflecting normal GBA.

In certain embodiments, GBA agonists may increase GBA mRNA by at least about 5% compared to a control. More preferably the GBA agonists may increase GBA mRNA by at least about 10%, such as at least about 15%, at least about 20%, at least about 25%, at least about 30%, at least about 35%, at least about 40%, at least about 45%, at least about 50%, at least about 60%, at least about 70%, at least about 80%, at least about 90%, at least about 100%, at least about 200%, at least about 300%, at least about 400%, at least about 500%, at least about 600%, or more compared to a control.

In certain embodiments, GBA agonists may increase GBA protein by at least about 5% compared to a control. More preferably GBA agonists may increase GBA protein by at least about 10%, such as at least about 15%, at least about 20%, at least about 25%, at least about 30%, at least about 35%, at least about 40%, at least about 45%, at least about 50%, at least about 60%, at least about 70%, at least about 80%, at least about 90%, at least about 100%, at least about 200%, at least about 300%, at least about 400%, at least about 500%, at least about 600%, or more compared to a control.

In certain embodiments the GBA agonists may increase GBA mRNA and protein by at least about 5% compared to a control. More preferably the GBA agonists may increase GBA mRNA and protein by at least about 10%, such as at least about 15%, at least about 20%, at least about 25%, at least about 30%, at least about 35%, at least about 40%, at least about 45%, at least about 50%, at least about 60%, at least about 70%, at least about 80%, at least about 90%, at least about 100%, at least about 200%, at least about 300%, at least about 400%, at least about 500%, at least about 600%, or more compared to a control.

Particularly contemplated GBA agonists of the invention are oligonucleotides of 8 to 40 nucleotides in length, or conjugates thereof, which comprise a contiguous nucleotide sequence complementary to at least 6, such as at least 7, such as at least 8 contiguous bases in the 3′ untranslated region (UTR) of an RNA sequence encoding GBA. The RNA sequence encoding GBA is preferably an mRNA sequence comprising the sequence of SEQ ID NO:73 or an allelic variant thereof.

### Oligonucleotide

The term “oligonucleotide” as used herein is defined, as is generally understood by the skilled person, as a molecule comprising two or more covalently linked nucleosides. Such covalently bound nucleosides may also be referred to as nucleic acid molecules or oligomers.

Oligonucleotides are commonly made in a laboratory by solid-phase chemical synthesis followed by purification and isolation. When referring to the sequence of an oligonucleotide, reference is made to the sequence or order of nucleobase moieties, or modifications thereof, of the covalently linked nucleotides or nucleosides. The oligonucleotides of the invention are man-made, and are chemically synthesized, and are typically purified or isolated. The oligonucleotides of the invention may comprise one or more modified nucleosides such as 2′ sugar modified nucleosides. The oligonucleotides of the invention may comprise one or more modified internucleoside linkages, such as one or more phosphorothioate internucleoside linkages.

Oligonucleotides according to the invention are capable of targeting the GBA mRNA transcript and can herein also be referred to as “antisense oligonucleotides.”

The oligonucleotides of the invention can be single stranded oligonucleotides or double-stranded oligonucleotides. In some preferred embodiments, the oligonucleotides of the invention are single-stranded oligonucleotides.

In some embodiments, the oligonucleotides of the invention are 8 to 40 nucleotides in length.

In some embodiments, the oligonucleotides of the invention are 8 to 40 nucleotides in length and comprise a contiguous nucleotide sequence of at least 6 nucleotides, such as 6 to 40 nucleotides.

In some embodiments, the oligonucleotides of the invention are 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39 or 40 nucleotides in length.

In some embodiments the oligonucleotides of the invention are at least 12 nucleotides in length.

In some embodiments the oligonucleotides of the invention are at least 14 nucleotides in length.

In some embodiments the oligonucleotides of the invention are at least 16 nucleotides in length.

In some embodiments the oligonucleotides of the invention are at least 18 nucleotides in length.

In some embodiments, the oligonucleotides of the invention are 16 to 20 nucleotides in length, such as 16, 18 or 20 nucleotides in length.

It is understood that the contiguous nucleotide sequence of the oligonucleotide cannot be longer than the oligonucleotide as such and that the oligonucleotide cannot be shorter than the contiguous nucleotide sequence.

In case of a double stranded oligonucleotide, the length measurement refers to the length of the strand comprising a contiguous nucleotide sequence complementary to at least 6 contiguous bases in the 3′ untranslated region (UTR) of an RNA sequence encoding glucocerebrosidase (GBA).

In some embodiments the oligonucleotide comprises the contiguous nucleotide sequence, and may optionally comprise further nucleotide(s), for example a nucleotide linker region which may be used to attach a functional group (e.g. a conjugate group) to the contiguous nucleotide sequence. The nucleotide linker region may or may not be complementary to the target nucleic acid.

### Contiguous Nucleotide Sequence

The term “contiguous nucleotide sequence” refers to the region of the oligonucleotide which is complementary to a target nucleic acid, which may be or may comprise an oligonucleotide motif sequence. The term is used interchangeably herein with the term “contiguous nucleobase sequence”.

The oligonucleotide comprises the contiguous nucleotide sequence, and may optionally comprise further nucleotide(s), for example a nucleotide linker region which may be used to attach a functional group (e.g. a conjugate group) to the contiguous nucleotide sequence. The nucleotide linker region may or may not be complementary to the target nucleic acid.

It is understood that the contiguous nucleotide sequence of the oligonucleotide cannot be longer than the oligonucleotide as such and that the oligonucleotide cannot be shorter than the contiguous nucleotide sequence.

In some embodiments, the entire nucleotide sequence of the oligonucleotide of the invention is the contiguous nucleotide sequence.

The contiguous nucleotide sequence is the sequence of nucleotides in the oligonucleotide of the invention which are complementary to, and in some instances fully complementary to, the target nucleic acid, target sequence, or target site sequence.

Target nucleic acids, target sequences, or target site sequences which can suitably be targeted by the oligonucleotides of the invention are described elsewhere wherein.

In some embodiments, the contiguous nucleotide sequence comprises a nucleobase sequence selected from the group consisting of SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO:11, SEQ ID NO:12, SEQ ID NO:13, SEQ ID NO:14, SEQ ID NO:15, SEQ ID NO:16, SEQ ID NO:17, SEQ ID NO:18, SEQ ID NO:19, SEQ ID NO:20, SEQ ID NO:21, SEQ ID NO:22, SEQ ID NO:23, SEQ ID NO:24, SEQ ID NO:25, SEQ ID NO:26, SEQ ID NO:27, SEQ ID NO:28, SEQ ID NO:29, SEQ ID NO:30, SEQ ID NO:31, SEQ ID NO:32, SEQ ID NO:33, and SEQ ID NO:34, or a fragment of at least 6 nucleotides of any thereof.

In some embodiments, the contiguous nucleotide sequence comprises a nucleobase sequence selected from the group consisting of SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO:11, SEQ ID NO:12, SEQ ID NO:13, SEQ ID NO:14, SEQ ID NO:15, SEQ ID NO:16, SEQ ID NO:17, SEQ ID NO:18, SEQ ID NO:19, SEQ ID NO:20, SEQ ID NO:21, SEQ ID NO:22, SEQ ID NO:23, SEQ ID NO:24, SEQ ID NO:25, SEQ ID NO:26, SEQ ID NO:27, SEQ ID NO:28, SEQ ID NO:29, SEQ ID NO:30, SEQ ID NO:31, SEQ ID NO:32, SEQ ID NO:33, and SEQ ID NO:34, or a fragment of at least 8 nucleotides of any thereof.

In some embodiments, the contiguous nucleotide sequence comprises a nucleobase sequence selected from the group consisting of SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO:11, SEQ ID NO:12, SEQ ID NO:13, SEQ ID NO:14, SEQ ID NO:15, SEQ ID NO:16, SEQ ID NO:17, SEQ ID NO:18, SEQ ID NO:19, SEQ ID NO:20, SEQ ID NO:21, SEQ ID NO:22, SEQ ID NO:23, SEQ ID NO:24, SEQ ID NO:25, SEQ ID NO:26, SEQ ID NO:27, SEQ ID NO:28, SEQ ID NO:29, SEQ ID NO:30, SEQ ID NO:31, SEQ ID NO:32, SEQ ID NO:33, and SEQ ID NO:34, or a fragment of at least 10 nucleotides of any thereof.

In some embodiments, the contiguous nucleotide sequence has a nucleobase sequence selected from the group consisting of SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO:11, SEQ ID NO:12, SEQ ID NO:13, SEQ ID NO:14, SEQ ID NO:15, SEQ ID NO:16, SEQ ID NO:17, SEQ ID NO:18, SEQ ID NO:19, SEQ ID NO:20, SEQ ID NO:21, SEQ ID NO:22, SEQ ID NO:23, SEQ ID NO:24, SEQ ID NO:25, SEQ ID NO:26, SEQ ID NO:27, SEQ ID NO:28, SEQ ID NO:29, SEQ ID NO:30, SEQ ID NO:31, SEQ ID NO:32, SEQ ID NO:33, and SEQ ID NO:34, or a fragment of at least 6 nucleotides of any thereof.

In some embodiments, the contiguous nucleotide sequence has a nucleobase sequence selected from the group consisting of SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO:11, SEQ ID NO:12, SEQ ID NO:13, SEQ ID NO:14, SEQ ID NO:15, SEQ ID NO:16, SEQ ID NO:17, SEQ ID NO:18, SEQ ID NO:19, SEQ ID NO:20, SEQ ID NO:21, SEQ ID NO:22, SEQ ID NO:23, SEQ ID NO:24, SEQ ID NO:25, SEQ ID NO:26, SEQ ID NO:27, SEQ ID NO:28, SEQ ID NO:29, SEQ ID NO:30, SEQ ID NO:31, SEQ ID NO:32, SEQ ID NO:33, and SEQ ID NO:34, or a fragment of at least 8 nucleotides of any thereof.

In some embodiments, the contiguous nucleotide sequence has a nucleobase sequence selected from the group consisting of SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO:11, SEQ ID NO:12, SEQ ID NO:13, SEQ ID NO:14, SEQ ID NO:15, SEQ ID NO:16, SEQ ID NO:17, SEQ ID NO:18, SEQ ID NO:19, SEQ ID NO:20, SEQ ID NO:21, SEQ ID NO:22, SEQ ID NO:23, SEQ ID NO:24, SEQ ID NO:25, SEQ ID NO:26, SEQ ID NO:27, SEQ ID NO:28, SEQ ID NO:29, SEQ ID NO:30, SEQ ID NO:31, SEQ ID NO:32, SEQ ID NO:33, and SEQ ID NO:34, or a fragment of at least 10 nucleotides of any thereof.

In some embodiments, the contiguous nucleotide sequence comprises a nucleobase sequence selected from the group consisting of SEQ ID NO:2, SEQ ID NO:3, SEQ ID NO:4, SEQ ID NO:5, SEQ ID NO:6, SEQ ID NO:7, SEQ ID NO:8, SEQ ID NO:10, SEQ ID NO:11, SEQ ID NO:12, SEQ ID NO:16, SEQ ID NO:19, SEQ ID NO:20, SEQ ID NO:21, SEQ ID NO:22, SEQ ID NO:23, SEQ ID NO:24, SEQ ID NO:25, SEQ ID NO:26, and SEQ ID NO:27.

In some embodiments, the contiguous nucleotide sequence has a nucleobase sequence selected from the group consisting of SEQ ID NO:2, SEQ ID NO:3, SEQ ID NO:4, SEQ ID NO:5, SEQ ID NO:6, SEQ ID NO:7, SEQ ID NO:8, SEQ ID NO:10, SEQ ID NO:11, SEQ ID NO:12, SEQ ID NO:16, SEQ ID NO:19, SEQ ID NO:20, SEQ ID NO:21, SEQ ID NO:22, SEQ ID NO:23, SEQ ID NO:24, SEQ ID NO:25, SEQ ID NO:26, and SEQ ID NO:27.

SEQ ID NOS:1 to 34 are RNA nucleobase sequences.

In some embodiments, the contiguous nucleotide sequence comprises the nucleobase sequence of SEQ ID NO:1.

In some embodiments, the contiguous nucleotide sequence comprises the nucleobase sequence of SEQ ID NO:2.

In some embodiments, the contiguous nucleotide sequence comprises the nucleobase sequence of SEQ ID NO:3.

In some embodiments, the contiguous nucleotide sequence comprises the nucleobase sequence of SEQ ID NO:4.

In some embodiments, the contiguous nucleotide sequence comprises the nucleobase sequence of SEQ ID NO:5.

In some embodiments, the contiguous nucleotide sequence comprises the nucleobase sequence of SEQ ID NO:6.

In some embodiments, the contiguous nucleotide sequence comprises the nucleobase sequence of SEQ ID NO:7.

In some embodiments, the contiguous nucleotide sequence comprises the nucleobase sequence of SEQ ID NO:8.

In some embodiments, the contiguous nucleotide sequence comprises the nucleobase sequence of SEQ ID NO:9.

In some embodiments, the contiguous nucleotide sequence comprises the nucleobase sequence of SEQ ID NO:10.

In some embodiments, the contiguous nucleotide sequence comprises the nucleobase sequence of SEQ ID NO:11.

In some embodiments, the contiguous nucleotide sequence comprises the nucleobase sequence of SEQ ID NO:12.

In some embodiments, the contiguous nucleotide sequence comprises the nucleobase sequence of SEQ ID NO:13.

In some embodiments, the contiguous nucleotide sequence comprises the nucleobase sequence of SEQ ID NO:14.

In some embodiments, the contiguous nucleotide sequence comprises the nucleobase sequence of SEQ ID NO:15.

In some embodiments, the contiguous nucleotide sequence comprises the nucleobase sequence of SEQ ID NO:16.

In some embodiments, the contiguous nucleotide sequence comprises the nucleobase sequence of SEQ ID NO:17.

In some embodiments, the contiguous nucleotide sequence comprises the nucleobase sequence of SEQ ID NO:18.

In some embodiments, the contiguous nucleotide sequence comprises the nucleobase sequence of SEQ ID NO:19.

In some embodiments, the contiguous nucleotide sequence comprises the nucleobase sequence of SEQ ID NO:20.

In some embodiments, the contiguous nucleotide sequence comprises the nucleobase sequence of SEQ ID NO:21.

In some embodiments, the contiguous nucleotide sequence comprises the nucleobase sequence of SEQ ID NO:22.

In some embodiments, the contiguous nucleotide sequence comprises the nucleobase sequence of SEQ ID NO:23.

In some embodiments, the contiguous nucleotide sequence comprises the nucleobase sequence of SEQ ID NO:24.

In some embodiments, the contiguous nucleotide sequence comprises the nucleobase sequence of SEQ ID NO:25.

In some embodiments, the contiguous nucleotide sequence comprises the nucleobase sequence of SEQ ID NO:26.

In some embodiments, the contiguous nucleotide sequence comprises the nucleobase sequence of SEQ ID NO:27.

In some embodiments, the contiguous nucleotide sequence comprises the nucleobase sequence of SEQ ID NO:28.

In some embodiments, the contiguous nucleotide sequence comprises the nucleobase sequence of SEQ ID NO:29.

In some embodiments, the contiguous nucleotide sequence comprises the nucleobase sequence of SEQ ID NO:30.

In some embodiments, the contiguous nucleotide sequence comprises the nucleobase sequence of SEQ ID NO:31.

In some embodiments, the contiguous nucleotide sequence comprises the nucleobase sequence of SEQ ID NO:32.

In some embodiments, the contiguous nucleotide sequence comprises the nucleobase sequence of SEQ ID NO:33.

In some embodiments, the contiguous nucleotide sequence comprises the nucleobase sequence of SEQ ID NO:34.

In some embodiments, the contiguous nucleotide sequence has the nucleobase sequence of SEQ ID NO:1 or a fragment thereof.

In some embodiments, the contiguous nucleotide sequence has the nucleobase sequence of SEQ ID NO:2 or a fragment thereof.

In some embodiments, the contiguous nucleotide sequence has the nucleobase sequence of SEQ ID NO:3 or a fragment thereof.

In some embodiments, the contiguous nucleotide sequence has the nucleobase sequence of SEQ ID NO:4 or a fragment thereof.

In some embodiments, the contiguous nucleotide sequence has the nucleobase sequence of SEQ ID NO:5 or a fragment thereof.

In some embodiments, the contiguous nucleotide sequence has the nucleobase sequence of SEQ ID NO:6 or a fragment thereof.

In some embodiments, the contiguous nucleotide sequence has the nucleobase sequence of SEQ ID NO:7 or a fragment thereof.

In some embodiments, the contiguous nucleotide sequence has the nucleobase sequence of SEQ ID NO:8 or a fragment thereof.

In some embodiments, the contiguous nucleotide sequence has the nucleobase sequence of SEQ ID NO:9 or a fragment thereof.

In some embodiments, the contiguous nucleotide sequence has the nucleobase sequence of SEQ ID NO:10 or a fragment thereof.

In some embodiments, the contiguous nucleotide sequence has the nucleobase sequence of SEQ ID NO: 11 or a fragment thereof.

In some embodiments, the contiguous nucleotide sequence has the nucleobase sequence of SEQ ID NO:12 or a fragment thereof.

In some embodiments, the contiguous nucleotide sequence has the nucleobase sequence of SEQ ID NO:13 or a fragment thereof.

In some embodiments, the contiguous nucleotide sequence has the nucleobase sequence of SEQ ID NO:14 or a fragment thereof.

In some embodiments, the contiguous nucleotide sequence has the nucleobase sequence of SEQ ID NO:15 or a fragment thereof.

In some embodiments, the contiguous nucleotide sequence has the nucleobase sequence of SEQ ID NO:16 or a fragment thereof.

In some embodiments, the contiguous nucleotide sequence has the nucleobase sequence of SEQ ID NO:17 or a fragment thereof.

In some embodiments, the contiguous nucleotide sequence has the nucleobase sequence of SEQ ID NO:18 or a fragment thereof.

In some embodiments, the contiguous nucleotide sequence has the nucleobase sequence of SEQ ID NO:19 or a fragment thereof.

In some embodiments, the contiguous nucleotide sequence has the nucleobase sequence of SEQ ID NO:20 or a fragment thereof.

In some embodiments, the contiguous nucleotide sequence has the nucleobase sequence of SEQ ID NO:21 or a fragment thereof.

In some embodiments, the contiguous nucleotide sequence has the nucleobase sequence of SEQ ID NO:22 or a fragment thereof.

In some embodiments, the contiguous nucleotide sequence has the nucleobase sequence of SEQ ID NO:23 or a fragment thereof.

In some embodiments, the contiguous nucleotide sequence has the nucleobase sequence of SEQ ID NO:24 or a fragment thereof.

In some embodiments, the contiguous nucleotide sequence has the nucleobase sequence of SEQ ID NO:25 or a fragment thereof.

In some embodiments, the contiguous nucleotide sequence has the nucleobase sequence of SEQ ID NO:26 or a fragment thereof.

In some embodiments, the contiguous nucleotide sequence has the nucleobase sequence of SEQ ID NO:27 or a fragment thereof.

In some embodiments, the contiguous nucleotide sequence has the nucleobase sequence of SEQ ID NO:28 or a fragment thereof.

In some embodiments, the contiguous nucleotide sequence has the nucleobase sequence of SEQ ID NO:29 or a fragment thereof.

In some embodiments, the contiguous nucleotide sequence has the nucleobase sequence of SEQ ID NO:30 or a fragment thereof.

In some embodiments, the contiguous nucleotide sequence has the nucleobase sequence of SEQ ID NO:31 or a fragment thereof.

In some embodiments, the contiguous nucleotide sequence has the nucleobase sequence of SEQ ID NO:32 or a fragment thereof.

In some embodiments, the contiguous nucleotide sequence has the nucleobase sequence of SEQ ID NO:33 or a fragment thereof.

In some embodiments, the contiguous nucleotide sequence has the nucleobase sequence of SEQ ID NO:34 or a fragment thereof.

In some embodiments the fragment may be at least 6, at least 7, at least 8, at least 9, at least 10, at least 11, at least 12, at least 13, at least 14, at least 15, at least 16, at least 17, at least 18 or at least 19 contiguous nucleotides of the contiguous nucleotide sequence. Preferably, the fragment is at least 6 contiguous nucleotides thereof, such as at least 8 contiguous nucleotides thereof, such as at least 10 contiguous nucleotides thereof.

In some embodiments, the contiguous nucleotide sequence is 6 to 40 nucleotides in length.

In some embodiments, the contiguous nucleotide sequence is 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39 or 40 nucleotides in length.

In some embodiments, the contiguous nucleotide sequence is 6 nucleotides in length.

In some embodiments, the contiguous nucleotide sequence is 8 nucleotides in length.

In some embodiments, the contiguous nucleotide sequence is 10 nucleotides in length.

In some embodiments the contiguous nucleotide sequence is 12 nucleotides in length.

In some embodiments, the contiguous nucleotide sequence is 14 nucleotides in length.

In some embodiments, the contiguous nucleotide sequence is 16 nucleotides in length.

In some embodiments, the contiguous nucleotide sequence is 18 nucleotides in length.

In a preferred embodiment, the contiguous nucleotide sequence is 16 to 20 nucleotides in length, such as 16, 18 or 20 nucleotides in length.

More preferably, the contiguous nucleotide sequence is 18 to 20 nucleotides in length.

In some embodiments, the contiguous nucleotide sequence is the same length as the antisense oligonucleotide.

In some embodiments, the oligonucleotide of the invention consists of the contiguous nucleotide sequence.

In some embodiments, the oligonucleotide of the invention is the contiguous nucleotide sequence.

For specific contiguous nucleotide sequences and oligonucleotides as disclosed herein, when the cytosine (C) residues are annotated as 5-methyl-cytosine (E), in various embodiments, one or more of the C residues present in the oligonucleotide may be unmodified C residues or modified cytosine residues other than E.

### Nucleotides and Nucleosides

Nucleotides and nucleosides are the building blocks of oligonucleotides and polynucleotides, and for the purposes of the present invention include both naturally occurring and non-naturally occurring nucleotides and nucleosides. In nature, nucleotides, such as DNA and RNA nucleotides, comprise a ribose sugar moiety, a nucleobase moiety and one or more phosphate groups (which is absent in nucleosides). Nucleosides and nucleotides may also interchangeably be referred to as “units” or “monomers”.

### Modified Nucleoside

The term “modified nucleoside” or “nucleoside modification” as used herein refers to nucleosides modified as compared to the equivalent DNA or RNA nucleoside by the introduction of one or more modifications of the sugar moiety or the (nucleo)base moiety.

Advantageously, an oligonucleotide according to the invention may comprise one or more modified nucleosides.

In some embodiments, the contiguous nucleobase sequence (motif sequence) can be modified to, for example, increase nuclease resistance and/or binding affinity to the target nucleic acid. Advantageously, high affinity modified nucleosides are used.

Advantageously, one or more of the modified nucleosides of the oligonucleotide according to the invention may comprise a modified sugar moiety. The term modified nucleoside may also be used herein interchangeably with the term “nucleoside analogue” or modified “units” or modified “monomers”. Nucleosides with an unmodified DNA or RNA sugar moiety are termed DNA or RNA nucleosides herein. Nucleosides with modifications in the base region of the DNA or RNA nucleoside are still generally termed DNA or RNA if they allow Watson Crick base pairing.

Exemplary modified nucleosides which may be used in the oligonucleotide according to the invention include, but are not limited to, 2′-O-alkyl-RNA, 2′-O-methyl-RNA, 2′-alkoxy-RNA, 2′-O-methoxyethyl-RNA, 2′-amino-DNA, 2′-fluoro-DNA, arabino nucleic acid (ANA), 2′-fluoro-ANA, morpholino and locked nucleic acid (LNA) nucleosides, such as from the group consisting of LNA, 2′-O-MOE, and 2′OMe nucleoside analogues.

In one embodiment, the oligonucleotide comprises one or more 2′-MOE RNA nucleosides.

In one embodiment, the oligonucleotide comprises one or more LNA nucleosides, such as beta-D-oxy-LNA nucleosides.

In one embodiment, the oligonucleotide comprises one or more 2′-O-methyl RNA nucleosides.

### Modified Internucleoside Linkage

Advantageously, the oligonucleotide according to the invention comprises one or more modified internucleoside linkages.

The term “modified internucleoside linkage” is defined as generally understood by the skilled person as linkages, other than phosphodiester (PO) linkages, which covalently couple two nucleosides together. The oligonucleotide of the invention may therefore comprise one or more modified internucleoside linkages such as one or more phosphorothioate internucleoside linkages.

In some embodiments at least 50% of the internucleoside linkages in the oligonucleotide according to the invention, or the contiguous nucleotide sequence thereof, are phosphorothioate, such as at least 60%, such as at least 70%, such as at least 75%, such as at least 80%, such as at least 90% or more. In some embodiments all of the internucleoside linkages of the oligonucleotide of the invention, or contiguous nucleotide sequence thereof, are phosphorothioate.

In a further embodiment, the oligonucleotide according to the invention comprises at least one modified internucleoside linkage. It is advantageous if at least 75%, such as all, of the internucleoside linkages within the contiguous nucleotide sequence are phosphorothioate or boranophosphate internucleoside linkages.

Advantageously, all the internucleoside linkages of the contiguous nucleotide sequence of the oligonucleotide according to the invention may be phosphorothioate, or all the internucleoside linkages of the oligonucleotide according to the invention may be phosphorothioate linkages.

### Nucleobase

The term “nucleobase” includes the purine (e.g. adenine and guanine) and pyrimidine (e.g. uracil, thymine and cytosine) moiety present in nucleosides and nucleotides which form hydrogen bonds in nucleic acid hybridization. In the context of the present invention the term nucleobase also encompasses modified nucleobases which may differ from naturally occurring nucleobases, but which are functional during nucleic acid hybridisation. In this context “nucleobase” refers to both naturally occurring nucleobases such as adenine, guanine, cytosine, thymidine, uracil, xanthine and hypoxanthine, as well as non-naturally occurring variants. Such variants are for example described in Hirao et al (2012) Accounts of Chemical Research vol 45 page 2055 and Bergstrom (2009) Current Protocols in Nucleic Acid Chemistry Suppl. 37 1.4.1.

In some embodiments, an oligonucleotide according to the invention comprises one or more of the modified nucleosides which are modified nucleobases.

In some embodiments, the nucleobase moiety is modified by changing the purine or pyrimidine into a modified purine or pyrimidine, such as substituted purine or substituted pyrimidine, such as a nucleobase selected from isocytosine, pseudoisocytosine, 5-methyl cytosine, 5-thiozolo-cytosine, 5-propynyl-cytosine, 5-propynyl-uracil, 5-bromouracil 5-thiazolo-uracil, 2-thio-uracil, 2′thio-thymine, PPG (7-Deaza-8-aza-dG-CE Phosphoramidite), inosine, diaminopurine, 6-aminopurine, 2-aminopurine, 2,6-diaminopurine and 2-chloro-6-aminopurine.

The nucleobase moieties may be indicated by the letter code for each corresponding nucleobase, e.g. A, T, G, C or U, wherein each letter may optionally include modified nucleobases of equivalent function. For example, in the exemplified oligonucleotides, the nucleobase moieties are selected from A, T, G, C, and 5-methyl cytosine. Optionally, for LNA gapmers, 5-methyl cytosine LNA nucleosides may be used. 5-methyl cytosine may be denoted as “E”.

Unless otherwise indicated or contradicted by context, in the present disclosure, oligonucleotides or target sequences which are RNA sequences can be presented herein with thymine (T) nucleobases designating uracil (U) nucleobases.

### Modified Oligonucleotide

The oligonucleotide according to the invention may be a modified oligonucleotide.

The term “modified oligonucleotide” describes an oligonucleotide comprising one or more sugar-modified nucleosides and/or modified internucleoside linkages. The term “chimeric oligonucleotide” is a term that has been used in the literature to describe oligonucleotides comprising sugar modified nucleosides and DNA nucleosides. In some embodiments, it may be advantageous for the oligonucleotide according to the invention to be a chimeric oligonucleotide.

In some embodiments, the oligonucleotide according to the invention, or contiguous nucleotide sequence thereof, may include modified nucleobases, which function as the shown nucleobase in base pairing, for example 5-methyl cytosine may be used in place of methyl cytosine. Inosine may be used as a universal base.

It is understood that the contiguous nucleobase sequences (motif sequence) can be modified to, for example, increase nuclease resistance and/or binding affinity to the target nucleic acid.

The pattern in which the modified nucleosides (such as high affinity modified nucleosides) are incorporated into the oligonucleotide sequence is generally termed oligonucleotide design.

In an embodiment, the oligonucleotide according to the invention comprises at least 1 modified nucleoside, such as at least 2, at least 3, at least 4, at least 5, at least 6, at least 7, at least 8, at least 9, at least 10, at least 11, at least 12, at least 13, at least 14, at least 15, at least 16, at least 17, at least 18, or at least 19 modified nucleosides.

Suitable modifications are described herein under the headings “modified nucleoside”, “high affinity modified nucleosides”, “sugar modifications”, “2′ sugar modifications” and “Locked nucleic acids (LNA)”.

### High Affinity Modified Nucleosides

A high affinity modified nucleoside is a modified nucleoside which, when incorporated into an oligonucleotide, enhances the affinity of the oligonucleotide for its complementary target, for example as measured by the melting temperature (T^{m}). A high affinity modified nucleoside of the present invention preferably results in an increase in melting temperature between +0.5 to +12° C., more preferably between +1.5 to +10° C. and most preferably between +3 to +8° C. per modified nucleoside. Numerous high affinity modified nucleosides are known in the art and include for example, many 2′ substituted nucleosides as well as locked nucleic acids (LNA) (see e.g. Freier & Altmann, Nucl. Acid Res., 1997, 25, 4429-4443 and Uhlmann; Curr. Opinion in Drug Development, 2000, 3(2), 293-213).

### Sugar Modifications

The oligonucleotide according to the invention may comprise one or more nucleosides which have a modified sugar moiety, i.e. a modification of the sugar moiety when compared to the ribose sugar moiety found in DNA and RNA.

Numerous nucleosides with modification of the ribose sugar moiety have been made, primarily with the aim of improving certain properties of oligonucleotides, such as affinity and/or nuclease resistance.

Such modifications include those where the ribose ring structure is modified, e.g. by replacement with a hexose ring (HNA), or a bicyclic ring, which typically have a biradicle bridge between the C2 and C4 carbons on the ribose ring (LNA), or an unlinked ribose ring which typically lacks a bond between the C2 and C3 carbons (e.g. UNA). Other sugar modified nucleosides include, for example, bicyclohexose nucleic acids (WO2011/017521) or tricyclic nucleic acids (WO2013/154798). Modified nucleosides also include nucleosides where the sugar moiety is replaced with a non-sugar moiety, for example in the case of peptide nucleic acids (PNA), or morpholino nucleic acids.

Sugar modifications also include modifications made via altering the substituent groups on the ribose ring to groups other than hydrogen, or the 2′-OH group naturally found in DNA and RNA nucleosides. Substituents may, for example be introduced at the 2′, 3′, 4′ or 5′ positions.

### 2′ Sugar Modified Nucleosides

A 2′ sugar modified nucleoside is a nucleoside which has a substituent other than H or —OH at the 2′ position (2′ substituted nucleoside) or comprises a 2′ linked biradicle capable of forming a bridge between the 2′ carbon and a second carbon in the ribose ring, such as LNA (2′-4′ biradical bridged) nucleosides.

Numerous 2′ substituted nucleosides have been found to have beneficial properties when incorporated into oligonucleotides. For example, a 2′ modified sugar may provide enhanced binding affinity and/or increased nuclease resistance to the oligonucleotide. Examples of 2′ substituted modified nucleosides are 2′-O-alkyl-RNA, 2′-O-methyl-RNA (2′oMe), 2′-alkoxy-RNA, 2′-O-methoxyethyl-RNA (MOE), 2′-amino-DNA, 2′-Fluoro-RNA, and 2′-F-ANA nucleoside. For further examples, please see e.g. Freier & Altmann; Nucl. Acid Res., 1997, 25, 4429-4443 and Uhlmann; Curr. Opinion in Drug Development, 2000, 3(2), 203-213, and Deleavey and Damha, Chemistry and Biology 2012, 19, 937. Illustrations of some 2′ substituted modified nucleosides are shown in Scheme 1.

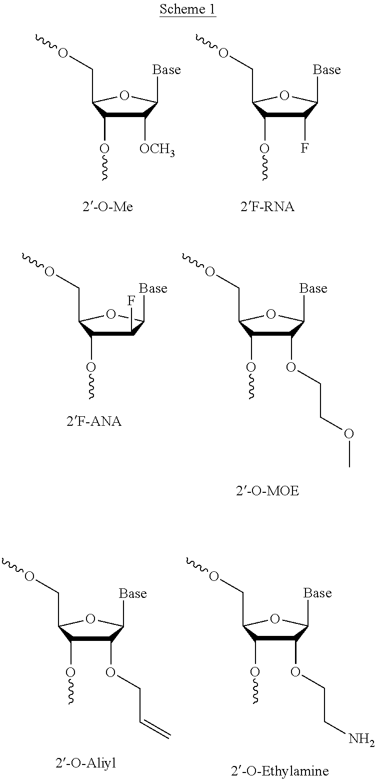

In relation to the present invention, 2′ substituted sugar modified nucleosides does not include 2 bridged nucleosides like LNA.

In one embodiment, the oligonucleotide according to the invention comprises one or more sugar modified nucleosides, such as 2′ sugar modified nucleosides.

Preferably, the oligonucleotide according to the invention comprises one or more 2′ sugar modified nucleosides independently selected from the group consisting of 2′-O-alkyl-RNA, 2′-O-methyl-RNA (2′oMe), 2′-alkoxy-RNA, 2′-O-methoxyethyl-RNA (2′MOE), 2′-amino-DNA, 2′-fluoro-DNA, arabino nucleic acid (ANA), and 2′-fluoro-ANA nucleosides, such as selected from 2′MOE and 2′oMe.

Locked Nucleic Acid Nucleosides (LNA nucleoside)

An “LNA nucleoside” is a modified nucleoside which comprises a biradical linking the C2′ and C4′ of the ribose sugar ring of said nucleoside (also referred to as a “2′-4′ bridge”), which restricts or locks the conformation of the ribose ring. These nucleosides are also termed bridged nucleic acid or bicyclic nucleic acid (BNA) in the literature. The locking of the conformation of the ribose is associated with an enhanced affinity of hybridization (duplex stabilization) when the LNA is incorporated into an oligonucleotide for a complementary RNA or DNA molecule. This can be routinely determined by measuring the melting temperature of the oligonucleotide/complement duplex.

Non limiting, exemplary LNA nucleosides are disclosed in WO 99/014226, WO 00/66604, WO 98/039352, WO 2004/046160, WO 00/047599, WO 2007/134181, WO 2010/077578, WO 2010/036698, WO 2007/090071, WO 2009/006478, WO 2011/156202, WO 2008/154401, WO 2009/067647, WO 2008/150729, Morita et al., Bioorganic & Med. Chem. Lett. 12, 73-76, Seth et al. J. Org. Chem. 2010, Vol 75(5) pp. 1569-81, and Mitsuoka et al., Nucleic Acids Research 2009, 37(4), 1225-1238, and Wan and Seth, J. Medical Chemistry 2016, 59, 9645-9667.

Further non-limiting, exemplary LNA nucleosides are disclosed in Scheme 2.

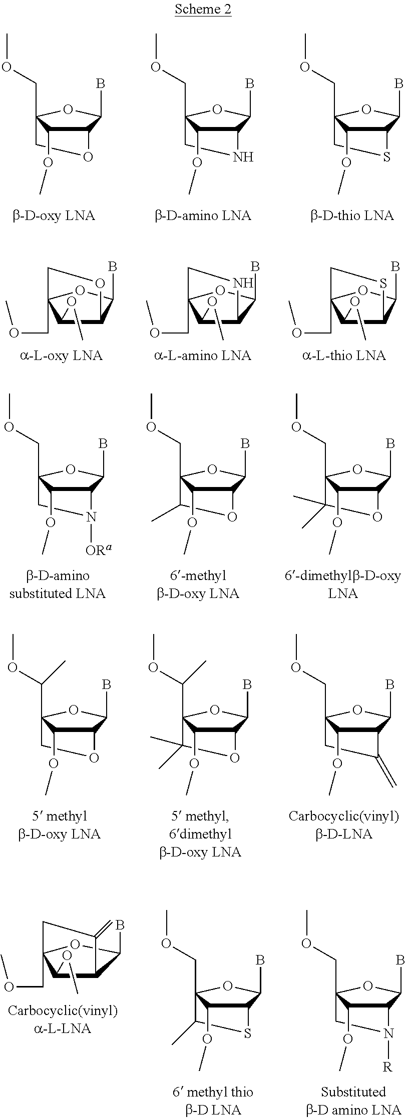

Particular LNA nucleosides are beta-D-oxy-LNA, 6′-methyl-beta-D-oxy LNA such as (S)-6′-methyl-beta-D-oxy-LNA (ScET) and ENA.

A particularly advantageous LNA is beta-D-oxy-LNA.

### Morpholino Oligonucleotides

In some embodiments, the oligonucleotide capable of increasing the expression of OBA of the invention comprises or consists of morpholino nucleosides (i.e. is a Morpholino oligomer and as a phosphorodiamidate Morpholino oligomer (PMO)). Splice-modulating morpholino oligonucleotides have been approved for clinical use—see for example eteplirsen, a 30 nt morpholino oligonucleotide targeting a frame shift mutation in DMD, used to treat Duchenne muscular dystrophy. Morpholino oligonucleotides have nucleobases attached to six membered morpholino rings rather than to ribose, such as methylenemorpholine rings linked through phosphorodiamidate groups, for example as illustrated by the following illustration of 4 consecutive morpholino nucleotides:

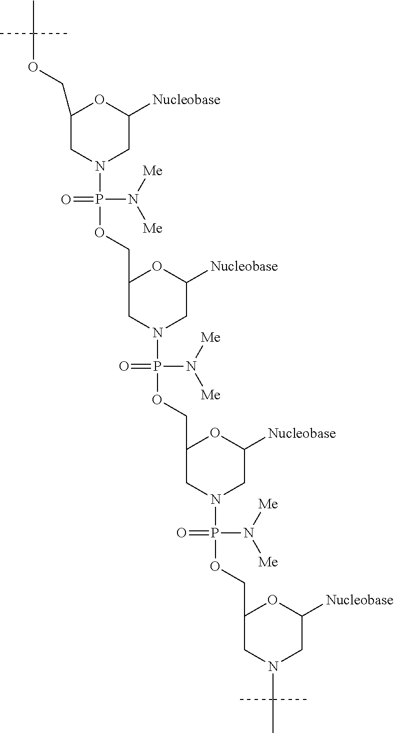

In some embodiments, morpholino oligonucleotides according to the invention may be, for example 8 to 40 morpholino nucleotides in length, such as 16 to 20 morpholino nucleotides in length, such as 18 to 20 morpholino nucleotides in length.

### RNase H Activity and Recruitment

The RNase H activity of an oligonucleotide refers to its ability to recruit RNase H when in a duplex with a complementary RNA molecule. WO 01/23613 provides in vitro methods for determining RNase H activity, which may be used to determine the ability to recruit RNase H.

Typically an oligonucleotide is deemed capable of recruiting RNase H if it, when provided with a complementary target nucleic acid sequence, has an initial rate, as measured in pmol/1/min, of at least 5%, such as at least 10%, at least 20% or more than 20%, of the initial rate determined when using an oligonucleotide having the same base sequence as the modified oligonucleotide being tested, but containing only DNA monomers with phosphorothioate linkages between all monomers in the oligonucleotide, and using the methodology provided by Examples 91-95 of WO 01/23613 (hereby incorporated by reference). For use in determining RHase H activity, recombinant RNase H1 is available from Lubio Science GmbH, Lucerne, Switzerland.

DNA oligonucleotides are known to effectively recruit RNase H, as are gapmer oligonucleotides which comprise a region of DNA nucleosides (typically at least 5 or 6 contiguous DNA nucleosides), flanked 5′ and 3′ by regions comprising 2′ sugar modified nucleosides, typically high affinity 2′ sugar modified nucleosides, such as 2-O-MOE and/or LNA.

For effectively increasing GBA expression, degradation of the GBA mRNA is not desirable, and as such it is preferable to avoid RNaseH-mediated degradation of the target. Therefore, the oligonucleotides of the invention are not RNase H recruiting oligonucleotides such as a gapmer oligonucleotide. RNase H recruitment may be avoided by limiting the number of contiguous DNA nucleotides in the oligonucleotide. Instead, oligonucleotide designs which do not recruit RNase H, e.g., mixmers and totalmers as described herein, may be used.

Advantageously, the oligonucleotides of the invention, or the contiguous nucleotide sequence thereof, comprise at most 4 contiguous DNA nucleosides, such as at most 3 contiguous DNA nucleosides, such as at most 2 contiguous DNA nucleosides, such as at most 1 DNA nucleosides, or no DNA nucleoside.

### Mixmers and Totalmers

Mixmers and totalmers are steric blocking oligonucleotide designs which do not recruit RNase H.

A mixmer is an oligonucleotide or a contiguous nucleotide sequence thereof which comprises at least one type of modified nucleosides. For example, a mixmer may comprise sugar modified nucleosides, such as 2′ sugar modified nucleosides, and short regions of DNA nucleosides, such as 1, 2 or 3 DNA nucleosides. Non-limiting examples of mixmer designs include every second design, wherein the nucleosides alternate between 1 LNA and 1 DNA nucleoside, e.g. LDLDLDLDLDLDLDLL, with 5′ and 3′ terminal LNA nucleosides, and every third design, such as LDDLDDLDDLDDLDDL, where every third nucleoside is a LNA nucleoside. Alternatively, a mixmer may comprise a mixture of modified nucleosides, such as MLMLMLMLMLMLMLMLMLML, wherein L=LNA and M=2′-O-MOE nucleosides. Other mixmer designs include 18-mers comprising fifteen 2′-O-MOE nucleosides and, at the 3′ end, three LNAs.

A totalmer is an oligonucleotide or a contiguous nucleotide sequence thereof which does not comprise DNA or RNA nucleosides, and may for example comprise only 2′-O-MOE nucleosides, such as a fully MOE phosphorothioate, e.g. MMMMMMMMMMMMMMMMMMMM, where M=2′-O-MOE, or may for example comprise only 2′oMe nucleosides.

Advantageously, the internucleoside nucleosides in mixmers and totalmers may be phosphorothioate, or a majority of nucleoside linkages in mixmers may be phosphorothioate. Mixmers and totalmers may also or alternatively comprise other internucleoside linkages, such as phosphodiester or phosphorodithioate, by way of example.

In some embodiments, an oligonucleotide of the invention is, or comprises, a mixmer or totalmer.

In some embodiments, an oligonucleotide of the invention is, or comprises, a mixmer.

In some embodiments, an oligonucleotide of the invention is, or comprises, a totalmer.

In some embodiments, the contiguous nucleotide sequence of an oligonucleotide of the invention is a mixmer or a tolalmer.

In some embodiments, the contiguous nucleotide sequence of an oligonucleotide of the invention is a mixmer.

In some embodiments, the contiguous nucleotide sequence of an oligonucleotide of the invention is a tolalmer.

### Target Sequence

The oligonucleotides of the invention target the 3′ untranslated region (UTR) of the GBA mRNA transcript, preferably the human GBA mRNA transcript. This may also be referred to herein as the GBA mRNA. GBA RNA sequences or segments thereof may herein also be referred to as target sequences, target nucleic acids or target site sequences.

GBA mRNA sequences include any and all naturally occurring mRNA sequences which encode GBA protein, such as a human GBA protein. GBA mRNA sequences are transcribed from GBA genomic sequences, such as human GBA genomic sequences. In some embodiments, the human GBA gene has the sequence of NCBI Reference NG_009783.1. In some embodiments, the human GBA genomic sequence corresponds to ENSG00000177628 (chr1: 155234452-155244699, reverse strand.GRCh38:CM000663.2) (SEQ ID NO:72). In some embodiments, the GBA mRNA has the nucleotide sequence set forth in SEQ ID NO:73, corresponding to ENST00000368373.8. However, it is to be understood that the GBA mRNA sequences contemplated as target sequences include those transcribed from allelic variants of the human GBA genomic sequence, such as allelic variants which comprise one or more polymorphisms.

In the GBA mRNA sequences, the target sequence is located in the 3′ UTR downstream of the stop codon TAG, which is located at positions 1746 to 1748 in SEQ ID NO:73. The oligonucleotides of the invention particularly target segments of the 3′ UTR of the GBA mRNA which are binding sites for a miR selected from miR-22-3p and a variant thereof. Preferably, the variant comprises a seed region fully complementary to GGCAGCT, except for one or two mismatches, such as one mismatch. Preferred target sequences in the 3′ UTR of the GBA mRNA are located in proximity to GGCAGCT, which is located, as indicated, at positions 2259 to 2265 in SEQ ID NO:73. Particularly contemplated are target sequences located in a GBA mRNA segment corresponding to the segment defined by positions 2227 to 2274 of SEQ ID NO:73.

In some embodiments, the target sequence is selected from the group of GBA mRNA segments defined by the following positions in a GBA genomic sequence corresponding to ENSG00000177628 or SEQ ID NO:72: 10184 to 10201, 10184 to 10201, 10185 to 10202, 10186 to 10203, 10187 to 10204, 10188 to 10205, 10189 to 10206, 10190 to 10207, 10191 to 10208, 10192 to 10209, 10193 to 10210, 10194 to 10211, 10195 to 10212, 10196 to 10213, 10197 to 10214, 10198 to 10215, 10199 to 10216, 10200 to 10217, 10201 to 10218, 10202 to 10219, 10203 to 10220, 10204 to 10221, 10205 to 10222, 10205 to 10222, 10206 to 10223, 10207 to 10224, 10208 to 10225, 10209 to 10226, 10210 to 10227, 10211 to 10228, 10212 to 10229, 10213 to 10230, and 10214 to 10231.

Unless otherwise stated or contradicted by context, all ranges herein are inclusive of the start and end value.

In some embodiments, the target sequence is or comprises an RNA sequence selected from SEQ ID NO:36; SEQ ID NO:37; SEQ ID NO:38; SEQ ID NO:39; SEQ ID NO:40; SEQ ID NO:41; SEQ ID NO:42; SEQ ID NO:43; SEQ ID NO:44; SEQ ID NO:45; SEQ ID NO:46; SEQ ID NO:47; SEQ ID NO:48; SEQ ID NO:49; SEQ ID NO:50; SEQ ID NO:51; SEQ ID NO:52; SEQ ID NO:53; SEQ ID NO:54; SEQ ID NO:55; SEQ ID NO:56; SEQ ID NO:57; SEQ ID NO:58; SEQ ID NO:59; SEQ ID NO:60; SEQ ID NO:61; SEQ ID NO:62; SEQ ID NO:63; SEQ ID NO:64; SEQ ID NO:65; SEQ ID NO:66; SEQ ID NO:67; and SEQ ID NO:68 (for nucleobase sequences, see Table 1).

Accordingly, in some embodiments, the oligonucleotide of the invention comprises a contiguous nucleotide sequence which is at least 75%, at least 80%, at least 85%, at least 90%, at least 95%, or fully, complementary to contiguous bases located downstream of the stop codon TAG at positions 1746 to 1748 in SEQ ID NO:73.

In some embodiments, the oligonucleotide of the invention comprises a contiguous nucleotide sequence which is at least 80%, at least 85%, at least 90%, at least 95%, or fully, complementary to a miR-22-3p binding site located downstream of the stop codon TAG at positions 1746 to 1748 in SEQ ID NO:73. Preferably, the miR-22-3p binding site is located in proximity to, or comprises, the nucleotide sequence corresponding to GGCAGCT.

In some embodiments, the oligonucleotide of the invention comprises a contiguous nucleotide sequence which is at least 75%, at least 80%, at least 85%, at least 90%, at least 91%, at least 92%, at least 93%, at least 94%, at least 95%, at least 96%, at least 97%, at least 98%, at least 99%, or fully, complementary to contiguous bases located in the segment at positions 2227 to 2274 in SEQ ID NO:73.

In one embodiment, the RNA sequence encoding GBA is an mRNA sequence comprising a 3′UTR sequence comprising the contiguous bases of positions 2227 to 2274 of SEQ ID NO:73.

In one embodiment, the RNA sequence encoding GBA is an mRNA sequence comprising the sequence of SEQ ID NO:73 or an allelic variant thereof.

In some embodiments, the oligonucleotide of the invention comprises a contiguous nucleotide sequence which is complementary to a target nucleic acid sequence selected from the group consisting of SEQ ID NO:35, SEQ ID NO:36, SEQ ID NO:37, SEQ ID NO:38, SEQ ID NO:39, SEQ ID NO:40, SEQ ID NO:41, SEQ ID NO:42, SEQ ID NO:43, SEQ ID NO:44, SEQ ID NO:45, SEQ ID NO:46, SEQ ID NO:47, SEQ ID NO:48, SEQ ID NO:49, SEQ ID NO:50, SEQ ID NO:51, SEQ ID NO:52, SEQ ID NO:53, SEQ ID NO:54, SEQ ID NO:55, SEQ ID NO:56, SEQ ID NO:57, SEQ ID NO:58, SEQ ID NO:59, SEQ ID NO:60, SEQ ID NO:61, SEQ ID NO:62, SEQ ID NO:63, SEQ ID NO:64, SEQ ID NO:65, SEQ ID NO:66, SEQ ID NO:67, SEQ ID NO:68, or to a fragment of at least 6 nucleotides of any thereof.

In some embodiments, the oligonucleotide of the invention comprises a contiguous nucleotide sequence which is complementary to a target nucleic acid sequence selected from the group consisting of SEQ ID NO:35, SEQ ID NO:36, SEQ ID NO:37, SEQ ID NO:38, SEQ ID NO:39, SEQ ID NO:40, SEQ ID NO:41, SEQ ID NO:42, SEQ ID NO:43, SEQ ID NO:44, SEQ ID NO:45, SEQ ID NO:46, SEQ ID NO:47, SEQ ID NO:48, SEQ ID NO:49, SEQ ID NO:50, SEQ ID NO:51, SEQ ID NO:52, SEQ ID NO:53, SEQ ID NO:54, SEQ ID NO:55, SEQ ID NO:56, SEQ ID NO:57, SEQ ID NO:58, SEQ ID NO:59, SEQ ID NO:60, SEQ ID NO:61, SEQ ID NO:62, SEQ ID NO:63, SEQ ID NO:64, SEQ ID NO:65, SEQ ID NO:66, SEQ ID NO:67, SEQ ID NO:68, or to a fragment of at least 8 nucleotides of any thereof.

In some embodiments, the oligonucleotide of the invention comprises a contiguous nucleotide sequence which is complementary to a target nucleic acid sequence selected from the group consisting of SEQ ID NO:35, SEQ ID NO:36, SEQ ID NO:37, SEQ ID NO:38, SEQ ID NO:39, SEQ ID NO:40, SEQ ID NO:41, SEQ ID NO:42, SEQ ID NO:43, SEQ ID NO:44, SEQ ID NO:45, SEQ ID NO:46, SEQ ID NO:47, SEQ ID NO:48, SEQ ID NO:49, SEQ ID NO:50, SEQ ID NO:51, SEQ ID NO:52, SEQ ID NO:53, SEQ ID NO:54, SEQ ID NO:55, SEQ ID NO:56, SEQ ID NO:57, SEQ ID NO:58, SEQ ID NO:59, SEQ ID NO:60, SEQ ID NO:61, SEQ ID NO:62, SEQ ID NO:63, SEQ ID NO:64, SEQ ID NO:65, SEQ ID NO:66, SEQ ID NO:67, SEQ ID NO:68, or to a fragment of at least 10 nucleotides of any thereof.

In some embodiments, the contiguous nucleotide sequence is complementary, such as at least 75%, at least 80%, at least 85%, at least 90%, at least 95%, or fully complementary, to a target nucleic acid sequence which comprises SEQ ID NO:35.

In some embodiments, the contiguous nucleotide sequence is complementary, such as at least 75%, at least 80%, at least 85%, at least 90%, at least 95%, or fully complementary, to a target nucleic acid sequence which comprises SEQ ID NO:36.

In some embodiments, the contiguous nucleotide sequence is complementary, such as at least 75%, at least 80%, at least 85%, at least 90%, at least 95%, or fully complementary, to a target nucleic acid sequence which comprises SEQ ID NO:37.

In some embodiments, the contiguous nucleotide sequence is complementary, such as at least 75%, at least 80%, at least 85%, at least 90%, at least 95%, or fully complementary, to a target nucleic acid sequence which comprises SEQ ID NO:38.

In some embodiments, the contiguous nucleotide sequence is complementary, such as at least 75%, at least 80%, at least 85%, at least 90%, at least 95%, or fully complementary, to a target nucleic acid sequence which comprises SEQ ID NO:39.

In some embodiments, the contiguous nucleotide sequence is complementary, such as at least 75%, at least 80%, at least 85%, at least 90%, at least 95%, or fully complementary, to a target nucleic acid sequence which comprises SEQ ID NO:40.

In some embodiments, the contiguous nucleotide sequence is complementary, such as at least 75%, at least 80%, at least 85%, at least 90%, at least 95%, or fully complementary, to a target nucleic acid sequence which comprises SEQ ID NO:41.

In some embodiments, the contiguous nucleotide sequence is complementary, such as at least 75%, at least 80%, at least 85%, at least 90%, at least 95%, or fully complementary, to a target nucleic acid sequence which comprises SEQ ID NO:42.

In some embodiments, the contiguous nucleotide sequence is complementary, such as at least 75%, at least 80%, at least 85%, at least 90%, at least 95%, or fully complementary, to a target nucleic acid sequence which comprises SEQ ID NO:43.

In some embodiments, the contiguous nucleotide sequence is complementary, such as at least 75%, at least 80%, at least 85%, at least 90%, at least 95%, or fully complementary, to a target nucleic acid sequence which comprises SEQ ID NO:44.

In some embodiments, the contiguous nucleotide sequence is complementary, such as at least 75%, at least 80%, at least 85%, at least 90%, at least 95%, or fully complementary, to a target nucleic acid sequence which comprises SEQ ID NO:45.

In some embodiments, the contiguous nucleotide sequence is complementary, such as at least 75%, at least 80%, at least 85%, at least 90%, at least 95%, or fully complementary, to a target nucleic acid sequence which comprises SEQ ID NO:46.

In some embodiments, the contiguous nucleotide sequence is complementary, such as at least 75%, at least 80%, at least 85%, at least 90%, at least 95%, or fully complementary, to a target nucleic acid sequence which comprises SEQ ID NO:47.

In some embodiments, the contiguous nucleotide sequence is complementary, such as at least 75%, at least 80%, at least 85%, at least 90%, at least 95%, or fully complementary, to a target nucleic acid sequence which comprises SEQ ID NO:48.

In some embodiments, the contiguous nucleotide sequence is complementary, such as at least 75%, at least 80%, at least 85%, at least 90%, at least 95%, or fully complementary, to a target nucleic acid sequence which comprises SEQ ID NO:49.

In some embodiments, the contiguous nucleotide sequence is complementary, such as at least 75%, at least 80%, at least 85%, at least 90%, at least 95%, or fully complementary, to a target nucleic acid sequence which comprises SEQ ID NO:50.

In some embodiments, the contiguous nucleotide sequence is complementary, such as at least 75%, at least 80%, at least 85%, at least 90%, at least 95%, or fully complementary, to a target nucleic acid sequence which comprises SEQ ID NO:51.

In some embodiments, the contiguous nucleotide sequence is complementary, such as at least 75%, at least 80%, at least 85%, at least 90%, at least 95%, or fully complementary, to a target nucleic acid sequence which comprises SEQ ID NO:52.

In some embodiments, the contiguous nucleotide sequence is complementary, such as at least 75%, at least 80%, at least 85%, at least 90%, at least 95%, or fully complementary, to a target nucleic acid sequence which comprises SEQ ID NO:53.

In some embodiments, the contiguous nucleotide sequence is complementary, such as at least 75%, at least 80%, at least 85%, at least 90%, at least 95%, or fully complementary, to a target nucleic acid sequence which comprises SEQ ID NO:54.

In some embodiments, the contiguous nucleotide sequence is complementary, such as at least 75%, at least 80%, at least 85%, at least 90%, at least 95%, or fully complementary, to a target nucleic acid sequence which comprises SEQ ID NO:55.

In some embodiments, the contiguous nucleotide sequence is complementary, such as at least 75%, at least 80%, at least 85%, at least 90%, at least 95%, or fully complementary, to a target nucleic acid sequence which comprises SEQ ID NO:56.

In some embodiments, the contiguous nucleotide sequence is complementary, such as at least 75%, at least 80%, at least 85%, at least 90%, at least 95%, or fully complementary, to a target nucleic acid sequence which comprises SEQ ID NO:57.

In some embodiments, the contiguous nucleotide sequence is complementary, such as at least 75%, at least 80%, at least 85%, at least 90%, at least 95%, or fully complementary, to a target nucleic acid sequence which comprises SEQ ID NO:58.

In some embodiments, the contiguous nucleotide sequence is complementary, such as at least 75%, at least 80%, at least 85%, at least 90%, at least 95%, or fully complementary, to a target nucleic acid sequence which comprises SEQ ID NO:59.

In some embodiments, the contiguous nucleotide sequence is complementary, such as at least 75%, at least 80%, at least 85%, at least 90%, at least 95%, or fully complementary, to a target nucleic acid sequence which comprises SEQ ID NO:60.

In some embodiments, the contiguous nucleotide sequence is complementary, such as at least 75%, at least 80%, at least 85%, at least 90%, at least 95%, or fully complementary, to a target nucleic acid sequence which comprises SEQ ID NO:61.

In some embodiments, the contiguous nucleotide sequence is complementary, such as at least 75%, at least 80%, at least 85%, at least 90%, at least 95%, or fully complementary, to a target nucleic acid sequence which comprises SEQ ID NO:62.

In some embodiments, the contiguous nucleotide sequence is complementary, such as at least 75%, at least 80%, at least 85%, at least 90%, at least 95%, or fully complementary, to a target nucleic acid sequence which comprises SEQ ID NO:63.

In some embodiments, the contiguous nucleotide sequence is complementary, such as at least 75%, at least 80%, at least 85%, at least 90%, at least 95%, or fully complementary, to a target nucleic acid sequence which comprises SEQ ID NO:64.

In some embodiments, the contiguous nucleotide sequence is complementary, such as at least 75%, at least 80%, at least 85%, at least 90%, at least 95%, or fully complementary, to a target nucleic acid sequence which comprises SEQ ID NO:65.

In some embodiments, the contiguous nucleotide sequence is complementary, such as at least 75%, at least 80%, at least 85%, at least 90%, at least 95%, or fully complementary, to a target nucleic acid sequence which comprises SEQ ID NO:66.

In some embodiments, the contiguous nucleotide sequence is complementary, such as at least 75%, at least 80%, at least 85%, at least 90%, at least 95%, or fully complementary, to a target nucleic acid sequence which comprises SEQ ID NO:67.

In some embodiments, the contiguous nucleotide sequence is complementary, such as at least 75%, at least 80%, at least 85%, at least 90%, at least 95%, or fully complementary, to a target nucleic acid sequence which comprises SEQ ID NO:68.

In some embodiments, the contiguous nucleotide sequence is fully complementary to a target nucleic acid sequence which comprises SEQ ID NO:35 or a fragment thereof.

In some embodiments, the contiguous nucleotide sequence is fully complementary to a target nucleic acid sequence which comprises SEQ ID NO:36 or a fragment thereof.

In some embodiments, the contiguous nucleotide sequence is fully complementary to a target nucleic acid sequence which comprises SEQ ID NO:37 or a fragment thereof.

In some embodiments, the contiguous nucleotide sequence is fully complementary to a target nucleic acid sequence which comprises SEQ ID NO:38 or a fragment thereof.

In some embodiments, the contiguous nucleotide sequence is fully complementary to a target nucleic acid sequence which comprises SEQ ID NO:39 or a fragment thereof.

In some embodiments, the contiguous nucleotide sequence is fully complementary to a target nucleic acid sequence which comprises SEQ ID NO:40 or a fragment thereof.

In some embodiments, the contiguous nucleotide sequence is fully complementary to a target nucleic acid sequence which comprises SEQ ID NO:41 or a fragment thereof.

In some embodiments, the contiguous nucleotide sequence is fully complementary to a target nucleic acid sequence which comprises SEQ ID NO:42 or a fragment thereof.

In some embodiments, the contiguous nucleotide sequence is fully complementary to a target nucleic acid sequence which comprises SEQ ID NO:43 or a fragment thereof.

In some embodiments, the contiguous nucleotide sequence is fully complementary to a target nucleic acid sequence which comprises SEQ ID NO:44 or a fragment thereof.

In some embodiments, the contiguous nucleotide sequence is fully complementary to a target nucleic acid sequence which comprises SEQ ID NO:45 or a fragment thereof.

In some embodiments, the contiguous nucleotide sequence is fully complementary to a target nucleic acid sequence which comprises SEQ ID NO:46 or a fragment thereof.

In some embodiments, the contiguous nucleotide sequence is fully complementary to a target nucleic acid sequence which comprises SEQ ID NO:47 or a fragment thereof.

In some embodiments, the contiguous nucleotide sequence is fully complementary to a target nucleic acid sequence which comprises SEQ ID NO:48 or a fragment thereof.

In some embodiments, the contiguous nucleotide sequence is fully complementary to a target nucleic acid sequence which comprises SEQ ID NO:49 or a fragment thereof.

In some embodiments, the contiguous nucleotide sequence is fully complementary to a target nucleic acid sequence which comprises SEQ ID NO:50 or a fragment thereof.

In some embodiments, the contiguous nucleotide sequence is fully complementary to a target nucleic acid sequence which comprises SEQ ID NO:51 or a fragment thereof.

In some embodiments, the contiguous nucleotide sequence is fully complementary to a target nucleic acid sequence which comprises SEQ ID NO:52 or a fragment thereof.

In some embodiments, the contiguous nucleotide sequence is fully complementary to a target nucleic acid sequence which comprises SEQ ID NO:53 or a fragment thereof.

In some embodiments, the contiguous nucleotide sequence is fully complementary to a target nucleic acid sequence which comprises SEQ ID NO:54 or a fragment thereof.

In some embodiments, the contiguous nucleotide sequence is fully complementary to a target nucleic acid sequence which comprises SEQ ID NO:55 or a fragment thereof.

In some embodiments, the contiguous nucleotide sequence is fully complementary to a target nucleic acid sequence which comprises SEQ ID NO:56 or a fragment thereof.

In some embodiments, the contiguous nucleotide sequence is fully complementary to a target nucleic acid sequence which comprises SEQ ID NO:57 or a fragment thereof.

In some embodiments, the contiguous nucleotide sequence is fully complementary to a target nucleic acid sequence which comprises SEQ ID NO:58 or a fragment thereof.

In some embodiments, the contiguous nucleotide sequence is fully complementary to a target nucleic acid sequence which comprises SEQ ID NO:59 or a fragment thereof.

In some embodiments, the contiguous nucleotide sequence is fully complementary to a target nucleic acid sequence which comprises SEQ ID NO:60 or a fragment thereof.

In some embodiments, the contiguous nucleotide sequence is fully complementary to a target nucleic acid sequence which comprises SEQ ID NO:61 or a fragment thereof.

In some embodiments, the contiguous nucleotide sequence is fully complementary to a target nucleic acid sequence which comprises SEQ ID NO:62 or a fragment thereof.

In some embodiments, the contiguous nucleotide sequence is fully complementary to a target nucleic acid sequence which comprises SEQ ID NO:63 or a fragment thereof.

In some embodiments, the contiguous nucleotide sequence is fully complementary to a target nucleic acid sequence which comprises SEQ ID NO:64 or a fragment thereof.

In some embodiments, the contiguous nucleotide sequence is fully complementary to a target nucleic acid sequence which comprises SEQ ID NO:65 or a fragment thereof.

In some embodiments, the contiguous nucleotide sequence is fully complementary to a target nucleic acid sequence which comprises SEQ ID NO:66 or a fragment thereof.

In some embodiments, the contiguous nucleotide sequence is fully complementary to a target nucleic acid sequence which comprises SEQ ID NO:67 or a fragment thereof.

In some embodiments, the contiguous nucleotide sequence is fully complementary to a target nucleic acid sequence which comprises SEQ ID NO:68 or a fragment thereof.

A fragment of the target sequence may be 6, 7, 8, 9, 10, 11, 12, 13, 14, 15, 16, or 17 nucleotides in length.

In some embodiments the contiguous nucleotide sequence is complementary to at least 6 contiguous nucleotides of any of the target sequences recited herein.

In some embodiments the contiguous nucleotide sequence is complementary to at least 8 contiguous nucleotides of any of the target sequences recited herein.

In some embodiments the contiguous nucleotide sequence is complementary to at least 10 contiguous nucleotides of any of the target sequences recited herein.

In some embodiments the contiguous nucleotide sequence is complementary to at least 12 contiguous nucleotides of any of the target sequences recited herein.

In some embodiments the contiguous nucleotide sequence is complementary to at least 14 contiguous nucleotides of any of the target sequences recited herein.

In some embodiments the contiguous nucleotide sequence is complementary to at least 16 contiguous nucleotides of any of the target sequences recited herein.

In some embodiments the contiguous nucleotide sequence is complementary to at least 18 contiguous nucleotides of any of the target sequences recited herein.

In some embodiments the contiguous nucleotide sequence is complementary to at least 19 contiguous nucleotides of any of the target sequences recited herein.

### Complementarity

The term “complementarity” describes the capacity for Watson-Crick base-pairing of nucleosides/nucleotides. Watson-Crick base pairs are guanine (G)-cytosine (C) and adenine (A)-thymine (T)/uracil (U).

It will be understood that oligonucleotides may comprise nucleosides with modified nucleobases, for example 5-methyl cytosine is often used in place of cytosine, and as such the term complementarity encompasses Watson Crick base-paring between non-modified and modified nucleobases (see for example Hirao et al (2012) Accounts of Chemical Research vol 45, page 2055 and Bergstrom (2009) Current Protocols in Nucleic Acid Chemistry Suppl. 37 1.4.1).

The term “% complementary” as used herein, refers to the proportion of nucleotides (in percent) of a contiguous nucleotide sequence in a nucleic acid molecule (e.g. oligonucleotide) which across the contiguous nucleotide sequence, are complementary to a reference sequence (e.g. a target sequence or sequence motif). The percentage of complementarity is thus calculated by counting the number of aligned nucleobases that are complementary (from Watson Crick base pairs) between the two sequences (when aligned with the target sequence 5′-3′ and the oligonucleotide sequence from 3′-5′), dividing that number by the total number of nucleotides in the oligonucleotide and multiplying by 100. In such a comparison a nucleobase/nucleotide which does not align (form a base pair) is termed a mismatch. Insertions and deletions are not allowed in the calculation of % complementarity of a contiguous nucleotide sequence. It will be understood that in determining complementarity, chemical modifications of the nucleobases are disregarded as long as the functional capacity of the nucleobase to form Watson Crick base pairing is retained (e.g. 5′-methyl cytosine is considered identical to a cytosine for the purpose of calculating % identity).

Within the present invention the term “complementary” requires the contiguous nucleotide sequence to be at least about 75% complementary, or at least about 80% complementarity, or at least about 85% complementarity, or at least about 90% complementary, or at least about 95% complementarity to a human GBA mRNA transcript. In some embodiments the contiguous nucleotide sequence may be at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98% or at least about 99% complementary to a human GBA mRNA transcript.

Put another way, for some embodiments, the contiguous nucleotide sequence of the oligonucleotide capable of increasing the expression of GBA according to the invention may include one, two, three, four, five or more mismatches, wherein a mismatch is a nucleotide within the contiguous nucleotide sequence which does not base pair with its target.

The term “fully complementary”, refers to 100% complementarity.

The oligonucleotide capable of increasing the expression of GBA according to the invention is complementary to a human GBA mRNA transcript, such as SEQ ID NO:73. It will be understood that the target GBA nucleic acid may be an allelic variant of SEQ ID NO:73, such as an allelic variant which comprises one or more polymorphism in the human GBA nucleic acid sequence.

### Identity

The term “identity” as used herein, refers to the proportion of nucleotides (expressed in percent) of a contiguous nucleotide sequence in a nucleic acid molecule (e.g. oligonucleotide) which across the contiguous nucleotide sequence, are identical to a reference sequence (e.g. a sequence motif).

The percentage of identity is thus calculated by counting the number of aligned nucleobases that are identical (a Match) between two sequences (in the contiguous nucleotide sequence of the compound of the invention and in the reference sequence), dividing that number by the total number of nucleotides in the oligonucleotide and multiplying by 100. Therefore, Percentage of Identity=(Matches×100)/Length of aligned region (e.g. the contiguous nucleotide sequence). Insertions and deletions are not allowed in the calculation the percentage of identity of a contiguous nucleotide sequence. It will be understood that in determining identity, chemical modifications of the nucleobases are disregarded as long as the functional capacity of the nucleobase to form Watson Crick base pairing is retained (e.g. 5-methyl cytosine is considered identical to a cytosine for the purpose of calculating % identity).

It is therefore to be understood that there is a relationship between identity and complementarity such that a contiguous nucleotide sequence within an oligonucleotide of the invention that is complementary to a target sequence also shares a percentage of identity with said complementary sequence.

### Hybridization

The terms “hybridising” or “hybridises” as used herein are to be understood as two nucleic acid strands (e.g. an oligonucleotide and a target nucleic acid) forming hydrogen bonds between base pairs on opposite strands thereby forming a duplex. The affinity of the binding between two nucleic acid strands is the strength of the hybridization. It is often described in terms of the melting temperature (Tm) defined as the temperature at which half of the oligonucleotides are duplexed with the target nucleic acid. At physiological conditions Tm is not strictly proportional to the affinity (Mergny and Lacroix, 2003, Oligonucleotides 13:515-537). The standard state Gibbs free energy ΔG° is a more accurate representation of binding affinity and is related to the dissociation constant (Kd) of the reaction by ΔG°=−RTIn(Kd), where R is the gas constant and T is the absolute temperature. Therefore, a very low ΔG° of the reaction between an oligonucleotide and the target nucleic acid reflects a strong hybridization between the oligonucleotide and target nucleic acid. ΔG° is the energy associated with a reaction where aqueous concentrations are 1M, the pH is 7, and the temperature is 37° C. The hybridization of oligonucleotides to a target nucleic acid is a spontaneous reaction and for spontaneous reactions ΔG° is less than zero. ΔG° can be measured experimentally, for example, by use of the isothermal titration calorimetry (ITC) method as described in Hansen et al., 1965, Chem. Comm. 36-38 and Holdgate et al., 2005, Drug Discov Today. The skilled person will know that commercial equipment is available for ΔG° measurements. ΔG° can also be estimated numerically by using the nearest neighbor model as described by SantaLucia, 1998, Proc Natl Acad Sci USA. 95: 1460-1465 using appropriately derived thermodynamic parameters described by Sugimoto et al., 1995, Biochemistry 34:11211-11216 and McTigue et al., 2004, Biochemistry 43:5388-5405.

In some embodiments, oligonucleotide of the present invention hybridises to a target nucleic acid with estimated ΔG° values below −10 kcal for oligonucleotides that are 10-30 nucleotides in length.

In some embodiments the degree or strength of hybridization is measured by the standard state Gibbs free energy ΔG°. The oligonucleotides of the invention may hybridise to a target nucleic acid with estimated ΔG° values below the range of −10 kcal, such as below −15 kcal, such as below −20 kcal and such as below −25 kcal. In certain embodiments the oligonucleotide of the invention hybridises to a sub-sequence of the target nucleic acid of SEQ ID NO: 1 with a ΔG° below −10 kcal, such as with a ΔG° between −10 to −60 kcal, such as −12 to −40, such as from −15 to −30 kcal or −16 to −27 kcal such as −18 to −25 kcal.

The GBA increase is enabled by the hybridisation between a contiguous nucleotide sequence of the oligonucleotide according to the invention and GBA mRNA. In some embodiments the oligonucleotide of the invention comprises mismatches between the oligonucleotide and the GBA mRNA. Despite mismatches, hybridisation to the target nucleic acid may still be sufficient to show a desired increase in GBA expression. If required, reduced binding affinity resulting from mismatches may advantageously be compensated by increasing the number of nucleotides in the oligonucleotide and/or an increasing the number of modified nucleosides capable of increasing the binding affinity to GBA mRNA, such as 2′ modified nucleosides or LNA, present within the oligonucleotide sequence.

### Antisense Oligonucleotides

The term “antisense oligonucleotide” as used herein is defined as an oligonucleotide capable hybridising to a target nucleic acid, in particular to a contiguous sequence on a target nucleic acid. Antisense oligonucleotides are not generally double stranded and are therefore not siRNAs or shRNAs.

The antisense oligonucleotides of the present invention may be single stranded. It is understood that single stranded oligonucleotides of the present invention can form hairpins or intermolecular duplex structures (duplex between two molecules of the same oligonucleotide), as long as the degree of intra or inter self-complementarity is less than approximately 50% across of the full length of the oligonucleotide.

In some embodiments, the single stranded antisense oligonucleotides of the invention may not contain RNA nucleosides.

Advantageously, the antisense oligonucleotides of the invention comprise one or more modified nucleosides or nucleotides, such as 2′ sugar modified nucleosides. Furthermore, in some antisense oligonucleotides of the invention, it may be advantageous that the nucleosides which are not modified are DNA nucleosides.

In some embodiments, the antisense oligonucleotide is 8 to 40 nucleotides in length.

In some embodiments, the antisense oligonucleotide is 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39 or 40 nucleotides in length.

In some embodiments the antisense oligonucleotide is at least 12 nucleotides in length.

In some embodiments the antisense oligonucleotide is at least 14 nucleotides in length.

In some embodiments the antisense oligonucleotide is at least 16 nucleotides in length.

In some embodiments the antisense oligonucleotide is at least 18 nucleotides in length.

In a preferred embodiment the antisense oligonucleotide is 16 to 20 nucleotides in length.

More preferably, the antisense oligonucleotide is 18 to 20 nucleotides in length.

In some embodiments the oligonucleotide of the invention is the antisense oligonucleotide.

Region D′ or D″ in an oligonucleotide The oligonucleotide of the invention may in some embodiments comprise or consist of the contiguous nucleotide sequence of the oligonucleotide which is complementary to the target nucleic acid, such as a mixmer or totalmer region, and further 5′ and/or 3′ nucleosides. The further 5′ and/or 3′ nucleosides may or may not be complementary, such as fully complementary, to the target nucleic acid. Such further 5′ and/or 3′ nucleosides may be referred to as region D′ and D″ herein.

The addition of region D′ or D″ may be used for the purpose of joining the contiguous nucleotide sequence, such as the mixmer or totalmer, to a conjugate moiety or another functional group. When used for joining the contiguous nucleotide sequence with a conjugate moiety is can serve as a biocleavable linker. Alternatively, it may be used to provide exonucleoase protection or for ease of synthesis or manufacture.

Region D′ or D″ may independently comprise or consist of 1, 2, 3, 4 or 5 additional nucleotides, which may be complementary or non-complementary to the target nucleic acid.

The D′ or D′ region may serve as a nuclease susceptible biocleavable linker (see definition of linkers). In some embodiments, the additional 5′ and/or 3′ end nucleotides are linked with phosphodiester linkages and are DNA or RNA. Nucleotide based biocleavable linkers suitable for use as region D′ or D″ are disclosed in WO 2014/076195, which include by way of example a phosphodiester linked DNA dinucleotide. The use of biocleavable linkers in poly-oligonucleotide constructs is disclosed in WO 2015/113922, where they are used to link multiple antisense constructs within a single oligonucleotide.

In one embodiment the oligonucleotide of the invention comprises a region D′ and/or D″ in addition to the contiguous nucleotide sequence which constitutes a mixmer or a totalmer.

In some embodiments the internucleoside linkage positioned between region D′ or D″ and the mixmer or totalmer region is a phosphodiester linkage.

### Conjugate

The invention encompasses an oligonucleotide capable of increasing the expression of GBA covalently attached to at least one conjugate moiety. In some embodiments this may be referred to as a conjugate of the invention.

The term “conjugate” as used herein refers to an oligonucleotide capable of increasing the expression of GBA which is covalently linked to a non-nucleotide moiety (conjugate moiety or region C or third region). The conjugate moiety may be covalently linked to the oligonucleotide of the invention optionally via a linker group, such as region D′ or D″.

Oligonucleotide conjugates and their synthesis has also been reported in comprehensive reviews by Manoharan in Antisense Drug Technology, Principles, Strategies, and Applications, S. T. Crooke, ed., Ch. 16, Marcel Dekker, Inc., 2001 and Manoharan, Antisense and Nucleic Acid Drug Development, 2002, 12, 103.

In some embodiments, the non-nucleotide moiety (conjugate moiety) is selected from the group consisting of carbohydrates (e.g. GalNAc), cell surface receptor ligands, drug substances, hormones, lipophilic substances, polymers, proteins, peptides, toxins (e.g. bacterial toxins), vitamins, viral proteins (e.g. capsids) or combinations thereof.

### Linkers

A linkage or linker is a connection between two atoms that links one chemical group or segment of interest to another chemical group or segment of interest via one or more covalent bonds. Conjugate moieties can be attached to the oligonucleotide capable of increasing the expression of GBA directly or through a linking moiety (e.g. linker or tether). Linkers serve to covalently connect a third region, e.g. a conjugate moiety (Region C), to a first region, e.g. an oligonucleotide or contiguous nucleotide sequence complementary to the target nucleic acid (region A).

In some embodiments of the invention the conjugate or oligonucleotide of the invention may optionally comprise a linker region (second region or region B and/or region Y) which is positioned between the oligonucleotide or contiguous nucleotide sequence complementary to the target nucleic acid (region A or first region) and the conjugate moiety (region C or third region).

Region B refers to biocleavable linkers comprising or consisting of a physiologically labile bond that is cleavable under conditions normally encountered or analogous to those encountered within a mammalian body. Conditions under which physiologically labile linkers undergo chemical transformation (e.g., cleavage) include chemical conditions such as pH, temperature, oxidative or reductive conditions or agents, and salt concentration found in or analogous to those encountered in mammalian cells. Mammalian intracellular conditions also include the presence of enzymatic activity normally present in a mammalian cell such as from proteolytic enzymes or hydrolytic enzymes or nucleases. In one embodiment the biocleavable linker is susceptible to S1 nuclease cleavage. In some embodiments the nuclease susceptible linker comprises between 1 and 5 nucleosides, such as DNA nucleoside(s) comprising at least two consecutive phosphodiester linkages. Phosphodiester containing biocleavable linkers are described in more detail in WO 2014/076195.

Region Y refers to linkers that are not necessarily biocleavable but primarily serve to covalently connect a conjugate moiety (region C or third region), to an oligonucleotide (region A or first region). The region Y linkers may comprise a chain structure or an oligomer of repeating units such as ethylene glycol, amino acid units or amino alkyl groups. The oligonucleotide conjugates of the present invention can be constructed of the following regional elements A-C, A-B-C, A-B-Y-C, A-Y-B-C or A-Y-C. In some embodiments the linker (region Y) is an amino alkyl, such as a C2-C36 amino alkyl group, including, for example C6 to C12 amino alkyl groups. In some embodiments the linker (region Y) is a C6 amino alkyl group.

### Salts

The term “salts” as used herein conforms to its generally known meaning, i.e. an ionic assembly of anions and cations.

The invention provides for pharmaceutically acceptable salts of the oligonucleotide according to the invention, or the conjugate according to the invention.

The invention provides for oligonucleotides according to the invention wherein the oligonucleotides are in the form of a pharmaceutically acceptable salt. In some embodiments the pharmaceutically acceptable salt may be a sodium salt or a potassium salt.

The invention provides for a pharmaceutically acceptable sodium salt of the oligonucleotide according to the invention.

The invention provides for a pharmaceutically acceptable potassium salt of the oligonucleotide according to the invention.

### Delivery of Oligonucleotide GBA Agonist

The invention provides for oligonucleotides according to the invention wherein the oligonucleotide is encapsulated in a lipid-based delivery vehicle, covalently linked to or encapsulated in a dendrimer, or conjugated to an aptamer.

This may be for the purpose of delivering the oligonucleotide of the invention to the targeted cells and/or to improve the pharmacokinetics of the oligonucleotide of the invention.

Examples of lipid-based delivery vehicles include oil-in-water emulsions, micelles, liposomes, and lipid nanoparticles.

### Pharmaceutical Compositions

In a further aspect, the invention provides pharmaceutical compositions comprising an oligonucleotide of the invention and a pharmaceutically acceptable diluent, carrier, salt and/or adjuvant. A pharmaceutically acceptable diluent includes phosphate-buffered saline (PBS) and pharmaceutically acceptable salts include, but are not limited to, sodium and potassium salts.

The invention provides for a pharmaceutical composition according to the invention, wherein the pharmaceutical composition comprises the oligonucleotide of the invention, and an aqueous diluent or solvent.

The invention provides for a solution, such as a phosphate buffered saline solution of the oligonucleotide of the invention. Suitably the solution, such as phosphate buffered saline solution, of the invention is a sterile solution.

WO 2007/031091 provides suitable and preferred examples of pharmaceutically acceptable diluents, carriers and adjuvants (hereby incorporated by reference). Suitable dosages, formulations, administration routes, compositions, dosage forms, combinations with other therapeutic agents, pro-drug formulations are also provided in WO 2007/031091.

Oligonucleotides of the invention may be mixed with pharmaceutically acceptable active or inert substances for the preparation of pharmaceutical compositions or formulations. Compositions and methods for the formulation of pharmaceutical compositions are dependent upon a number of criteria, including, but not limited to, route of administration, extent of disease, or dose to be administered.

In some embodiments, the oligonucleotide or oligonucleotide conjugate of the invention is a prodrug. In particular, with respect to oligonucleotide conjugates, the conjugate moiety of the oligonucleotide is cleaved once the prodrug is delivered to the site of action, e.g. the target cell.

### Target Cell

The term “target cell” as used herein refers to a cell which is expressing the target nucleic acid. In some embodiments the target cell may be in vivo or in vitro. In some embodiments the target cell is a mammalian cell such as a rodent cell, such as a mouse cell or a rat cell, or a primate cell such as a monkey cell or a human cell.

### Applications

The oligonucleotides of the invention may be utilised as, for example, therapeutic agents, including prophylactic agents, as well as research agents.

### Research Reagents

In research, oligonucleotides of the invention may be used to specifically increase the synthesis of GBA mRNA and/or protein in cells (e.g. in vitro cell cultures) and experimental animals, thereby facilitating functional analysis of the target or an appraisal of its usefulness as a target for therapeutic intervention.

### Method for Modulating GBA Expression

The invention provides for a method for enhancing, upregulating or restoring the expression of GBA in a cell, such as a cell which is expressing GBA, said method comprising contacting an oligonucleotide of the invention, or the pharmaceutical composition of the invention, in an effective amount with said cell.

In some embodiments the method is an in vitro method.

In some embodiments the method is an in vivo method.

In some embodiments, the cell is a mammalian cell, such as a human cell.

In some embodiments, the cell is part of, or derived from, a subject suffering from or susceptible to a disease associated with reduced expression of GBA. Such diseases include but are not limited Gaucher's disease, Parkinson's Disease, dementia, dementia with Lewy bodies (DLB) and rapid eye movements (REM) sleep behaviour disorders.

### Treatment

The term ‘treatment’ as used herein refers to both treatment of an existing disease (e.g. a disease or disorder as herein referred to), or prevention of a disease, i.e. prophylaxis. It will therefore be recognised that treatment as referred to herein may, in some embodiments, be prophylactic.

The invention provides methods for treating or preventing a disease, comprising administering a therapeutically or prophylactically effective amount of an oligonucleotide or a pharmaceutical composition of the invention to a subject suffering from or susceptible to the disease.

The invention provides for a method for treating or preventing a disease associated with reduced expression of GBA, comprising administering a therapeutically or prophylactically effective amount of an oligonucleotide of the invention or a pharmaceutical composition of the invention to a subject suffering from or susceptible to a disease associated with reduced expression of GBA.

In one embodiment, the disease is selected from the group consisting of Gaucher's disease, Parkinson's Disease, dementia, dementia with Lewy bodies (DLB) and rapid eye movements (REM) sleep behaviour disorders.

In one embodiment the disease is Parkinson's disease.

In one embodiment the disease is Gaucher's disease.

In some embodiments, the subject is an animal, preferably a mammal such as a mouse, rat, hamster, or monkey, or, most preferably, a human.

The invention provides for an oligonucleotide of the invention or a pharmaceutical composition of the invention, for use as a medicament.

An oligonucleotide of the invention, or a pharmaceutical composition of the invention, is typically administered in an effective amount.

The invention provides for the use of an oligonucleotide of the invention or a pharmaceutical composition of the invention for the preparation of a medicament.

The invention provides an oligonucleotide of the invention or a pharmaceutical composition according to the invention for use in therapy.

The methods of the invention are preferably employed for treatment, e.g., prophylactic treatment, of diseases caused by abnormal levels and/or activity of GBA. The disease may in particular be caused by reduced levels and/or activity of GBA protein.

The invention further relates to use of an oligonucleotide of the invention or a pharmaceutical composition of the invention as defined herein for the manufacture of a medicament for the treatment of abnormal levels and/or activity of GBA, in particular low levels and/or activity of GBA.

In one embodiment, the invention relates to an oligonucleotide of the invention or a pharmaceutical composition of the invention for use in the treatment of Gaucher's disease, Parkinson's disease dementia, dementia with Lewy bodies (DLB) and rapid eye movements (REM) sleep behaviour disorders.

The invention provides for the use of an oligonucleotide of the invention or a pharmaceutical composition of the invention, for the preparation of a medicament for the treatment or prevention of Gaucher's disease.

The invention provides for the use of an oligonucleotide of the invention or a pharmaceutical composition of the invention, for the preparation of a medicament for the treatment or prevention of Parkinson's disease.

### Administration

The oligonucleotide or pharmaceutical composition of the invention may be administered topically (such as, to the skin, inhalation, ophthalmic or otic) or enterally (such as, orally or through the gastrointestinal tract) or parenterally (such as, intravenous, subcutaneous, intramuscular, intracerebral, intracerebroventricular or intrathecal).

In a preferred embodiment the oligonucleotide of the invention is administered by a parenteral route including intravenous, intraarterial, subcutaneous, intraperitoneal or intramuscular injection or infusion, intrathecal or intracranial, e.g., intracerebral or intraventricular, administration. In one embodiment the oligonucleotide is administered intracerebrally or intracerebroventricularly. In another embodiment the oligonucleotide of the invention is administered intrathecally.

The invention also provides for the use of the oligonucleotide of the invention as described for the manufacture of a medicament wherein the medicament is in a dosage form for intrathecal administration.

The invention also provides for the use of the oligonucleotide of the invention as described for the manufacture of a medicament wherein the medicament is in a dosage form for intracerebral or intraventricular administration.

The invention also provides for the use of the oligonucleotide of the invention as described for the manufacture of a medicament wherein the medicament is in a dosage form for intracerebroventricular administration.

### Combination Therapies

In some embodiments the oligonucleotide or pharmaceutical composition of the invention is for use in a combination treatment with another therapeutic agent.

### Method of Manufacture

In a further aspect, the invention provides methods for manufacturing the oligonucleotides of the invention comprising reacting nucleotide units and thereby forming covalently linked contiguous nucleotide units comprised in the oligonucleotide. Preferably, the method uses phophoramidite WO 2017/081223 PCT/EP2016/077383 chemistry (see for example Caruthers et al, 1987, Methods in Enzymology vol. 154, pages 287-313).

In a further embodiment the method further comprises reacting the contiguous nucleotide sequence with a conjugating moiety (ligand). In a further aspect a method is provided for manufacturing the composition of the invention, comprising mixing the oligonucleotide or conjugated oligonucleotide of the invention with a pharmaceutically acceptable diluent, solvent, carrier, salt and/or adjuvant.

TABLE 1
Antisense oligonucleotide base and target site sequences
Exemplary compounds used in the Examples were designed as 18-mer blockmers
with a phosphorothioate backbone, some of which with 3 LNAs at the 3′ end
(see Table 2-Compound table). ″E″ indicates 5-methyl cytosine.

| | Base | | | Target | |
|---|---|---|---|---|---|
| | SEQ ID | Position in | Target Site | Site | Compound ID |
| Base sequence | NO | ENSG00000177628 | Sequence | SEQ ID NO | (See Table 2) |
| AGTTAEGCACCCAA | 1 | 8924 to 8937 | UUGGGUGCGUAACU | 35 | COMP ID NO 1 |
| GTGEEATAGGAATEA | 2 | 10184 to 10201 | AGAUGAUUCCUAUGGC | 36 | COMP ID NO 2 |
| GTGEEATAGGAATEA | 3 | 10184 to 10201 | AGAUGAUUCCUAUGGC | 37 | COMP ID NO 3 |
| GGTGEEATAGGAATE | 4 | 10185 to 10202 | GAUGAUUCCUAUGGCA | 38 | COMP ID NO 4 |
| TGGTGEEATAGGAAT | 5 | 10186 to 10203 | AUGAUUCCUAUGGCAC | 39 | COMP ID NO 5 |
| ETGGTGEEATAGGAA | 6 | 10187 to 10204 | UGAUUCCUAUGGCACC | 40 | COMP ID NO 6 |
| GETGGTGEEATAGGA | 7 | 10188 to 10205 | GAUUCCUAUGGCACCA | 41 | COMP ID NO 7 |
| GGETGGTGEEATAGG | 8 | 10189 to 10206 | AUUCCUAUGGCACCAG | 42 | COMP ID NO 8 |
| TGGETGGTGEEATAG | 9 | 10190 to 10207 | UUCCUAUGGCACCAGC | 43 | COMP ID NO 9 |
| ETGGETGGTGEEATA | 10 | 10191 to 10208 | UCCUAUGGCACCAGCC | 44 | COMP ID NO [symbol] |
| EETGGETGGTGEEAT | 11 | 10192 to 10209 | CCUAUGGCACCAGCCA | 45 | COMP ID NO [symbol] |
| TEETGGETGGTGEEA | 12 | 10193 to 10210 | CUAUGGCACCAGCCAG | 46 | COMP ID NO [symbol] |
| TTEETGGETGGTGEE | 13 | 10194 to 10211 | UAUGGCACCAGCCAGG | 47 | COMP ID NO [symbol] |
| TTTEETGGETGGTGE | 14 | 10195 to 10212 | AUGGCACCAGCCAGGA | 48 | COMP ID NO [symbol] |
| TTTTEETGGETGGTG | 15 | 10196 to 10213 | UGGCACCAGCCAGGAA | 49 | COMP ID NO [symbol] |
| TTTTTEETGGETGGT | 16 | 10197 to 10214 | GGCACCAGCCAGGAAA | 50 | COMP ID NO [symbol] |
| ATTTTTEETGGETGGT | 17 | 10198 to 10215 | GCACCAGCCAGGAAAA | 51 | COMP ID NO [symbol] |
| EATTTTTEETGGETGG | 18 | 10199 to 10216 | CACCAGCCAGGAAAAA | 52 | COMP ID NO [symbol] |
| EEATTTTTEETGGETG | 19 | 10200 to 10217 | ACCAGCCAGGAAAAAU | 53 | COMP ID NO [symbol] |
| GEEATTTTTEETGGET | 20 | 10201 to 10218 | CCAGCCAGGAAAAAUG | 54 | COMP ID NO [symbol] |
| TGEEATTTTTEETGGE | 21 | 10202 to 10219 | CAGCCAGGAAAAAUGG | 55 | COMP ID NO [symbol] |
| ETGEEATTTTTEETGG | 22 | 10203 to 10220 | AGCCAGGAAAAAUGGC | 56 | COMP ID NO [symbol] |
| GETGEEATTTTTEETG | 23 | 10204 to 10221 | GCCAGGAAAAAUGGCA | 57 | COMP ID NO [symbol] |
| AGETGEEATTTTTEET | 24 | 10205 to 10222 | CCAGGAAAAAUGGCAG | 58 | COMP ID NO [symbol] |
| AGETGEEATTTTTEET | 25 | 10205 to 10222 | CCAGGAAAAAUGGCAG | 59 | COMP ID NO [symbol] |
| GAGETGEEATTTTTEE | 26 | 10206 to 10223 | CAGGAAAAAUGGCAGC | 60 | COMP ID NO [symbol] |
| AGAGETGEEATTTTTE | 27 | 10207 to 10224 | AGGAAAAAUGGCAGCU | 61 | COMP ID NO [symbol] |
| AAGAGETGEEATTTTT | 28 | 10208 to 10225 | GGAAAAAUGGCAGCUC | 62 | COMP ID NO [symbol] |
| TAAGAGETGEEATTTT | 29 | 10209 to 10226 | GAAAAAUGGCAGCUCU | 63 | COMP ID NO [symbol] |
| TTAAGAGETGEEATTT | 30 | 10210 to 10227 | AAAAAUGGCAGCUCUU | 64 | COMP ID NO [symbol] |
| TTTAAGAGETGEEATT | 31 | 10211 to 10228 | AAAAUGGCAGCUCUUA | 65 | COMP ID NO [symbol] |
| ETTTAAGAGETGEEAT | 32 | 10212 to 10229 | AAAUGGCAGCUCUUAA | 66 | COMP ID NO [symbol] |
| EETTTAAGAGETGEE | 33 | 10213 to 10230 | AAUGGCAGCUCUUAAA | 67 | COMP ID NO [symbol] |
| TEETTTAAGAGETGEE | 34 | 10214 to 10231 | AUGGCAGCUCUUAAAG | 68 | COMP ID NO [symbol] |

[symbol]

indicates data missing or illegible when filed

TABLE 2
Compound Table
Helm Annotation Key:

[LR](G) is a beta-D-oxy-LNA guanine nucleoside,
[LR](T) is a beta-D-oxy-LNA thymine nucleoside,
[LR](A) is a beta-D-oxy-LNA adenine nucleoside,
[LR]([5meC] is a beta-D-oxy-LNA 5-methyl cytosine nucleoside,
[dR](G) is a DNA guanine nucleoside,
[dR](T) is a DNA thymine nucleoside,
[dR](A) is a DNA adenine nucleoside,
[dR]([C] is a DNA cytosine nucleoside,
[mR](G) is a 2′-O-methyl RNA guanine nucleoside,
[mR](U) is a 2′-O-methyl RNA DNA uracil nucleoside,
[mR](A) is a 2′-O-methyl RNA DNA adenine nucleoside,
[mR]([C] is a 2′-O-methyl RNA DNA cytosine nucleoside,
[sP] is a phosphorothioate internucleoside linkage.

Further details regarding how to read a HELM sequence are provided at www.pistoiaalliance.org/helm-tools/, accessed on 22 Dec. 2022.

| Compound | |
|---|---|
| ID | Helm, 5′ → 3′ direction |
| COMP ID | RNA1{[LR](A)[sP].[LR](G)[sP].[LR](T)[sP].[LR](T)[sP].[dR](A)[sP].[dR]([5meC])[sP].[dR](G)[sP].[dR](C)[sP].[dR](A)[sP].[dR] |
| NO 1 | (C)[sP].[dR](C)[sP].[dR](C)[sP].[LR](A)[sP].[LR](A)}$$$$V2.0 |
| COMP ID | RNA1{[MOE](G)[sP].[MOE](T)[sP].[MOE](G)[sP].[MOE]([5meC])[sP].[MOE]([5meC])[sP].[MOE](A)[sP].[MOE](T)[sP].[MOE] |
| NO 2 | (A)[sP].[MOE](G)[sP].[MOE](G)[sP].[MOE](A)[sP].[MOE](A)[sP].[MOE](T)[sP].[MOE]([5meC])[sP].[MOE](A)[sP].[LR](T) |
| | [sP].[LR]([5meC])[sP].[LR](T)}$$$$V2.0 |
| COMP ID | RNA1{[MOE](G)[sP].[MOE](T)[sP].[MOE](G)[sP].[MOE]([5meC])[sP].[MOE]([5meC])[sP].[MOE](A)[sP].[MOE](T)[sP].[MOE] |
| NO 3 | (A)[sP].[MOE](G)[sP].[MOE](G)[sP].[MOE](A)[sP].[MOE](A)[sP].[MOE](T)[sP].[MOE]([5meC])[sP].[MOE](A)[sP].[MOE](T) |
| | [sP].[MOE]([5meC])[sP].[MOE](T)}$$$$V2.0 |
| COMP ID | RNA1{[MOE](G)[sP].[MOE](G)[sP].[MOE](T)[sP].[MOE](G)[sP].[MOE]([5meC])[sP].[MOE]([5meC])[sP].[MOE](A)[sP].[MOE] |
| NO 4 | (T)[sP].[MOE](A)[sP].[MOE](G)[sP].[MOE](G)[sP].[MOE](A)[sP].[MOE](A)[sP].[MOE](T)[sP].[MOE]([5meC])[sP].[LR](A) |
| | [sP].[LR](T)[sP].[LR]([5meC])}$$$$V2.0 |
| COMP ID | RNA1{[MOE](T)[sP].[MOE](G)[sP].[MOE](G)[sP].[MOE](T)[sP].[MOE](G)[sP].[MOE]([5meC])[sP].[MOE]([5meC])[sP].[MOE] |
| NO 5 | (A)[sP].[MOE](T)[sP].[MOE](A)[sP].[MOE](G)[sP].[MOE](G)[sP].[MOE](A)[sP].[MOE](A)[sP].[MOE](T)[sP].[LR]([5meC]) |
| | [sP].[LR](A)[sP].[LR](T)}$$$$V2.0 |
| COMP ID | RNA1{[MOE]([5meC])[sP].[MOE](T)[sP].[MOE](G)[sP].[MOE](G)[sP].[MOE](T)[sP].[MOE](G)[sP].[MOE]([5meC])[sP].[MOE] |
| NO 6 | ([5meC])[sP].[MOE](A)[sP].[MOE](T)[sP].[MOE](A)[sP].[MOE](G)[sP].[MOE](G)[sP].[MOE](A)[sP].[MOE](A)[sP].[LR](T) |
| | [sP].[LR]([5meC])[sP].[LR](A)}$$$$V2.0 |
| COMP ID | RNA1{[MOE](G)[sP].[MOE]([5meC])[sP].[MOE](T)[sP].[MOE](G)[sP].[MOE](G)[sP].[MOE](T)[sP].[MOE](G)[sP].[MOE]([5meC]) |
| NO 7 | [sP].[MOE]([5meC])[sP].[MOE](A)[sP].[MOE](T)[sP].[MOE](A)[sP].[MOE](G)[sP].[MOE](G)[sP].[MOE](A)[sP].[LR](A) |
| | [sP].[LR](T)[sP].[LR]([5meC])}$$$$V2.0 |
| COMP ID | RNA1{[MOE](G)[sP].[MOE](G)[sP].[MOE]([5meC])[sP].[MOE](T)[sP].[MOE](G)[sP].[MOE](G)[sP].[MOE](T)[sP].[MOE](G) |
| NO 8 | [sP].[MOE]([5meC])[sP].[MOE]([5meC])[sP].[MOE](A)[sP].[MOE](T)[sP].[MOE](A)[sP].[MOE](G)[sP].[MOE](G)[sP].[LR](A) |
| | [sP].[LR](A)[sP].[LR](T)}$$$$V2.0 |
| COMP ID | RNA1{[MOE](T)[sP].[MOE](G)[sP].[MOE](G)[sP].[MOE]([5meC])[sP].[MOE](T)[sP].[MOE](G)[sP].[MOE](G)[sP].[MOE](T) |
| NO 9 | [sP].[MOE](G)[sP].[MOE]([5meC])[sP].[MOE]([5meC])[sP].[MOE](A)[sP].[MOE](T)[sP].[MOE](A)[sP].[MOE](G)[sP].[LR](G) |
| | [sP].[LR](A)[sP].[LR](A)}$$$$V2.0 |
| COMP ID | RNA1{[MOE]([5meC])[sP].[MOE](T)[sP].[MOE](G)[sP].[MOE](G)[sP].[MOE]([5meC])[sP].[MOE](T)[sP].[MOE](G)[sP].[MOE] |
| NO 10 | (G)[sP].[MOE](T)[sP].[MOE](G)[sP].[MOE]([5meC])[sP].[MOE]([5meC])[sP].[MOE](A)[sP].[MOE](T)[sP].[MOE](A)[sP].[LR] |
| | (G)[sP].[LR](G)[sP].[LR](A)}$$$$V2.0 |
| COMP ID | RNA1{[MOE]([5meC])[sP].[MOE]([5meC])[sP].[MOE](T)[sP].[MOE](G)[sP].[MOE](G)[sP].[MOE]([5meC])[sP].[MOE](T) |
| NO 11 | [sP].[MOE](G)[sP].[MOE](G)[sP].[MOE](T)[sP].[MOE](G)[sP].[MOE]([5meC])[sP].[MOE]([5meC])[sP].[MOE](A)[sP].[MOE] |
| | (T)[sP].[LR](A)[sP].[LR](G)[sP].[LR](G)}$$$$V2.0 |
| COMP ID | RNA1{[MOE](T)[sP].[MOE]([5meC])[sP].[MOE]([5meC])[sP].[MOE](T)[sP].[MOE](G)[sP].[MOE](G)[sP].[MOE]([5meC]) |
| NO 12 | [sP].[MOE](T)[sP].[MOE](G)[sP].[MOE](G)[sP].[MOE](T)[sP].[MOE](G)[sP].[MOE]([5meC])[sP].[MOE]([5meC])[sP].[MOE] |
| | (A)[sP].[LR](T)[sP].[LR](A)[sP].[LR](G)}$$$$V2.0 |
| COMP ID | RNA1{[MOE](T)[sP].[MOE](T)[sP].[MOE]([5meC])[sP].[MOE]([5meC])[sP].[MOE](T)[sP].[MOE](G)[sP].[MOE](G)[sP].[MOE] |
| NO 13 | ([5meC])[sP].[MOE](T)[sP].[MOE](G)[sP].[MOE](G)[sP].[MOE](T)[sP].[MOE](G)[sP].[MOE]([5meC])[sP].[MOE]([5meC]) |
| | [sP].[LR](A)[sP].[LR](T)[sP].[LR](A)}$$$$V2.0 |
| COMP ID | RNA1{[MOE](T)[sP].[MOE](T)[sP].[MOE](T)[sP].[MOE]([5meC])[sP].[MOE]([5meC])[sP].[MOE](T)[sP].[MOE](G)[sP].[MOE] |
| NO 14 | (G)[sP].[MOE]([5meC])[sP].[MOE](T)[sP].[MOE](G)[sP].[MOE](G)[sP].[MOE](T)[sP].[MOE](G)[sP].[MOE]([5meC])[sP].[LR] |
| | ([5meC])[sP].[LR](A)[sP].[LR](T)}$$$$V2.0 |
| COMP ID | RNA1{[MOE](T)[sP].[MOE](T)[sP].[MOE](T)[sP].[MOE](T)[sP].[MOE]([5meC])[sP].[MOE]([5meC])[sP].[MOE](T)[sP].[MOE] |
| NO 15 | (G)[sP].[MOE](G)[sP].[MOE]([5meC])[sP].[MOE](T)[sP].[MOE](G)[sP].[MOE](G)[sP].[MOE](T)[sP].[MOE](G)[sP].[LR] |
| | ([5meC])[sP].[LR]([5meC])[sP].[LR](A)}$$$$V2.0 |
| COMP ID | RNA1{[MOE](T)[sP].[MOE](T)[sP].[MOE](T)[sP].[MOE](T)[sP].[MOE](T)[sP].[MOE]([5meC])[sP].[MOE]([5meC])[sP].[MOE] |
| NO 16 | (T)[sP].[MOE](G)[sP].[MOE](G)[sP].[MOE]([5meC])[sP].[MOE](T)[sP].[MOE](G)[sP].[MOE](G)[sP].[MOE](T)[sP].[LR](G) |
| | [sP].[LR]([5meC])[sP].[LR]([5meC])}$$$$V2.0 |
| COMP ID | RNA1{[MOE](A)[sP].[MOE](T)[sP].[MOE](T)[sP].[MOE](T)[sP].[MOE](T)[sP].[MOE](T)[sP].[MOE]([5meC])[sP].[MOE] |
| NO 17 | ([5meC])[sP].[MOE](T)[sP].[MOE](G)[sP].[MOE](G)[sP].[MOE]([5meC])[sP].[MOE](T)[sP].[MOE](G)[sP].[MOE](G)[sP].[LR] |
| | (T)[sP].[LR](G)[sP].[LR]([5meC])}$$$$V2.0 |
| COMP ID | RNA1{[MOE]([5meC])[sP].[MOE](A)[sP].[MOE](T)[sP].[MOE](T)[sP].[MOE](T)[sP].[MOE](T)[sP].[MOE](T)[sP].[MOE] |
| NO 18 | ([5meC])[sP].[MOE]([5meC])[sP].[MOE](T)[sP].[MOE](G)[sP].[MOE](G)[sP].[MOE]([5meC])[sP].[MOE](T)[sP].[MOE](G) |
| | [sP].[LR](G)[sP].[LR](T)[sP].[LR](G)}$$$$V2.0 |
| COMP ID | RNA1{[MOE]([5meC])[sP].[MOE]([5meC])[sP].[MOE](A)[sP].[MOE](T)[sP].[MOE](T)[sP].[MOE](T)[sP].[MOE](T)[sP].[MOE] |
| NO 19 | (T)[sP].[MOE]([5meC])[sP].[MOE]([5meC])[sP].[MOE](T)[sP].[MOE](G)[sP].[MOE](G)[sP].[MOE]([5meC])[sP].[MOE](T) |
| | [sP].[LR](G)[sP].[LR](G)[sP].[LR](T)}$$$$V2.0 |
| COMP ID | RNA1{[MOE](G)[sP].[MOE]([5meC])[sP].[MOE]([5meC])[sP].[MOE](A)[sP].[MOE](T)[sP].[MOE](T)[sP].[MOE](T)[sP].[MOE] |
| NO 20 | (T)[sP].[MOE](T)[sP].[MOE]([5meC])[sP].[MOE]([5meC])[sP].[MOE](T)[sP].[MOE](G)[sP].[MOE](G)[sP].[MOE]([5meC]) |
| | [sP].[LR](T)[sP].[LR](G)[sP].[LR](G)}$$$$V2.0 |
| COMP ID | RNA1{[MOE](T)[sP].[MOE](G)[sP].[MOE]([5meC])[sP].[MOE]([5meC])[sP].[MOE](A)[sP].[MOE](T)[sP].[MOE](T)[sP].[MOE] |
| NO 21 | (T)[sP].[MOE](T)[sP].[MOE](T)[sP].[MOE]([5meC])[sP].[MOE]([5meC])[sP].[MOE](T)[sP].[MOE](G)[sP].[MOE](G)[sP].[LR] |
| | ([5meC])[sP].[LR](T)[sP].[LR](G)}$$$$V2.0 |
| COMP ID | RNA1{[MOE]([5meC])[sP].[MOE](T)[sP].[MOE](G)[sP].[MOE]([5meC])[sP].[MOE]([5meC])[sP].[MOE](A)[sP].[MOE](T) |
| NO 22 | [sP].[MOE](T)[sP].[MOE](T)[sP].[MOE](T)[sP].[MOE](T)[sP].[MOE]([5meC])[sP].[MOE]([5meC])[sP].[MOE](T)[sP].[MOE] |
| | (G)[sP].[LR](G)[sP].[LR]([5meC])[sP].[LR](T)}$$$$V2.0 |
| COMP ID | RNA1{[MOE](G)[sP].[MOE]([5meC])[sP].[MOE](T)[sP].[MOE](G)[sP].[MOE]([5meC])[sP].[MOE]([5meC])[sP].[MOE](A) |
| NO 23 | [sP].[MOE](T)[sP].[MOE](T)[sP].[MOE](T)[sP].[MOE](T)[sP].[MOE](T)[sP].[MOE]([5meC])[sP].[MOE]([5meC])[sP].[MOE] |
| | (T)[sP].[LR](G)[sP].[LR](G)[sP].[LR]([5meC])}$$$$V2.0 |
| COMP ID | RNA1{[MOE](A)[sP].[MOE](G)[sP].[MOE]([5meC])[sP].[MOE](T)[sP].[MOE](G)[sP].[MOE]([5meC])[sP].[MOE]([5meC]) |
| NO 24 | [sP].[MOE](A)[sP].[MOE](T)[sP].[MOE](T)[sP].[MOE](T)[sP].[MOE](T)[sP].[MOE](T)[sP].[MOE]([5meC])[sP].[MOE] |
| | ([5meC])[sP].[LR](T)[sP].[LR](G)[sP].[LR](G)}$$$$V2.0 |
| COMP ID | RNA1{[MOE](A)[sP].[MOE](G)[sP].[MOE]([5meC])[sP].[MOE](T)[sP].[MOE](G)[sP].[MOE]([5meC])[sP].[MOE]([5meC]) |
| NO 25 | [sP].[MOE](A)[sP].[MOE](T)[sP].[MOE](T)[sP].[MOE](T)[sP].[MOE](T)[sP].[MOE](T)[sP].[MOE]([5meC])[sP].[MOE] |
| | ([5meC])[sP].[MOE](T)[sP].[MOE](G)[sP].[MOE](G)}$$$$V2.0 |
| COMP ID | RNA1{[MOE](G)[sP].[MOE](A)[sP].[MOE](G)[sP].[MOE]([5meC])[sP].[MOE](T)[sP].[MOE](G)[sP].[MOE]([5meC])[sP].[MOE] |
| NO 26 | ([5meC])[sP].[MOE](A)[sP].[MOE](T)[sP].[MOE](T)[sP].[MOE](T)[sP].[MOE](T)[sP].[MOE](T)[sP].[MOE]([5meC])[sP].[LR] |
| | ([5meC])[sP].[LR](T)[sP].[LR](G)}$$$$V2.0 |
| COMP ID | RNA1{[MOE](A)[sP].[MOE](G)[sP].[MOE](A)[sP].[MOE](G)[sP].[MOE]([5meC])[sP].[MOE](T)[sP].[MOE](G)[sP].[MOE] |
| NO 27 | ([5meC])[sP].[MOE]([5meC])[sP].[MOE](A)[sP].[MOE](T)[sP].[MOE](T)[sP].[MOE](T)[sP].[MOE](T)[sP].[MOE](T)[sP].[LR] |
| | ([5meC])[sP].[LR]([5meC])[sP].[LR](T)}$$$$V2.0 |
| COMP ID | RNA1{[MOE](A)[sP].[MOE](A)[sP].[MOE](G)[sP].[MOE](A)[sP].[MOE](G)[sP].[MOE]([5meC])[sP].[MOE](T)[sP].[MOE](G) |
| NO 28 | [sP].[MOE]([5meC])[sP].[MOE]([5meC])[sP].[MOE](A)[sP].[MOE](T)[sP].[MOE](T)[sP].[MOE](T)[sP].[MOE](T)[sP].[LR](T) |
| | [sP].[LR]([5meC])[sP].[LR]([5meC])}$$$$V2.0 |
| COMP ID | RNA1{[MOE](T)[sP].[MOE](A)[sP].[MOE](A)[sP].[MOE](G)[sP].[MOE](A)[sP].[MOE](G)[sP].[MOE]([5meC])[sP].[MOE](T) |
| NO 29 | [sP].[MOE](G)[sP].[MOE]([5meC])[sP].[MOE]([5meC])[sP].[MOE](A)[sP].[MOE](T)[sP].[MOE](T)[sP].[MOE](T)[sP].[LR] |
| | (T)[sP].[LR](T)[sP].[LR]([5meC])}$$$$V2.0 |
| COMP ID | RNA1{[MOE](T)[sP].[MOE](T)[sP].[MOE](A)[sP].[MOE](A)[sP].[MOE](G)[sP].[MOE](A)[sP].[MOE](G)[sP].[MOE]([5meC]) |
| NO 30 | [sP].[MOE](T)[sP].[MOE](G)[sP].[MOE]([5meC])[sP].[MOE]([5meC])[sP].[MOE](A)[sP].[MOE](T)[sP].[MOE](T)[sP].[LR] |
| | (T)[sP].[LR](T)[sP].[LR](T)}$$$$V2.0 |
| COMP ID | RNA1{[MOE](T)[sP].[MOE](T)[sP].[MOE](T)[sP].[MOE](A)[sP].[MOE](A)[sP].[MOE](G)[sP].[MOE](A)[sP].[MOE](G) |
| NO 31 | [sP].[MOE]([5meC])[sP].[MOE](T)[sP].[MOE](G)[sP].[MOE]([5meC])[sP].[MOE]([5meC])[sP].[MOE](A)[sP].[MOE](T)[sP].[LR] |
| | (T)[sP].[LR](T)[sP].[LR](T)}$$$$V2.0 |
| COMP ID | RNA1{[MOE]([5meC])[sP].[MOE](T)[sP].[MOE](T)[sP].[MOE](T)[sP].[MOE](A)[sP].[MOE](A)[sP].[MOE](G)[sP].[MOE](A) |
| NO 32 | [sP].[MOE](G)[sP].[MOE]([5meC])[sP].[MOE](T)[sP].[MOE](G)[sP].[MOE]([5meC])[sP].[MOE]([5meC])[sP].[MOE](A)[sP].[LR] |
| | (T)[sP].[LR](T)[sP].[LR](T)}$$$$V2.0 |
| COMP ID | RNA1{[MOE]([5meC])[sP].[MOE]([5meC])[sP].[MOE](T)[sP].[MOE](T)[sP].[MOE](T)[sP].[MOE](A)[sP].[MOE](A)[sP].[MOE] |
| NO 33 | (G)[sP].[MOE](A)[sP].[MOE](G)[sP].[MOE]([5meC])[sP].[MOE](T)[sP].[MOE](G)[sP].[MOE]([5meC])[sP].[MOE]([5meC]) |
| | [sP].[LR](A)[sP].[LR](T)[sP].[LR](T)}$$$$V2.0 |
| COMP ID | RNA1{[MOE](T)[sP].[MOE]([5meC])[sP].[MOE]([5meC])[sP].[MOE](T)[sP].[MOE](T)[sP].[MOE](T)[sP].[MOE](A)[sP].[MOE] |
| NO 34 | (A)[sP].[MOE](G)[sP].[MOE](A)[sP].[MOE](G)[sP].[MOE]([5meC])[sP].[MOE](T)[sP].[MOE](G)[sP].[MOE]([5meC])[sP].[LR] |
| | ([5meC])[sP].[LR](A)[sP].[LR](T)}$$$$V2.0 |
| | [Intentionally blank] |
| | [Intentionally blank] |

### EXAMPLES

### Example 1: Upregulation of GBA mRNA

The day before transfection, H4 glioblastoma cells were plated to a density of 10000 per well in 96 well plates in full growth medium (DMEM Sigma: D0819, 10% FBS, 1 mM Sodium Pyruvate). The day after plating, the cells were either transfected with GBA targeting antisense oligonucleotides COMP ID NO 2 to 34 (n=2), a GBA targeting gapmer (COMP ID NO:1, or PBS (Mock) using Lipofectamin RNAiMax (Invitrogen) at a final concentration of 5 and 25 nM according to the manufacturer's instructions. 48 h after transfection, mRNA was isolated using the RNeasy® 96 Kit (Qiagen) and extracted in 200 μL RNAse free Water. 4 μL was used as input for one-step RT-qPCR analysis according to protocol in Table 3, (qScript™ XLT One-Step RT-qPCR ToughMix®, Low ROX™, Quanta Bioscience, #95134-500) using custom designed qPCR assay specific to GBA (Table 4) and predesigned assay for TBP (HEX, Hs.PT.58v.39858774, IDT). GBA mRNA concentrations were quantified relative to the housekeeping gene TBP.

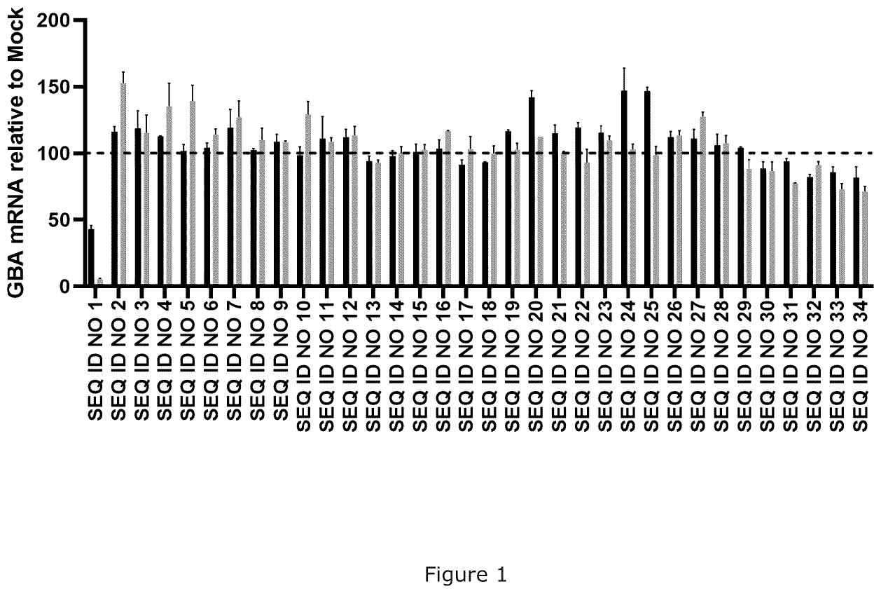

The results are shown in FIG. 1. COMP ID NO 2, COMP ID NO 3, COMP ID NO 4, COMP ID NO 5, COMP ID NO 6, COMP ID NO 7, COMP ID NO 8, COMP ID NO 10, COMP ID NO 11, COMP ID NO 12, COMP ID NO 16, COMP ID NO 19, COMP ID NO 20, COMP ID NO 21, COMP ID NO 22, COMP ID NO 23, COMP ID NO 24, COMP ID NO 25, COMP ID NO 26, and COMP ID NO 27 all increased expression of GBA mRNA more than 10% relative to Mock 48 h after transfection in H4 cells.

TABLE 3
GBA gPCR assay: Primers and probe,
all sequences 5′ → 3′.

| | | |
|---|---|---|
| GBA | Primer 1 | CCAATTGGGTGCGTAACTTTG |
| e9_10 | | (SEQ ID NO:69) |
| | Primer 2 | GTTCTTCTGACTGGCAACCA |
| | | (SEQ ID NO:70) |
| | Probe | 56-FAM/ACAGCCCAT/ZEN/ |
| | | GTTCTACCACCTTGG/3IAbkFQ |
| | | (SEQ ID NO:71) |

In Table 3, ZEN and 3IAbkFQ represent quenchers. The ZEN™ internal quencher has a structure proprietary to Integrated DNA Technologies, Inc. (IDT). 3IAbkFQ is an Iowa Black® quencher from IDT.

TABLE 4
RT-qPCR protocol

| Temperature | Time | Cycles |
|---|---|---|
| 50° C. | 15:00 min | |
| 95° C. | 03:20 min | |
| 95° C. | 00:05 min | 40x |
| 60° C. | 00:45 min | |

Temperature change rate 1.9° C./sec for all steps

1. An antisense oligonucleotide of 8 to 40 nucleotides in length, which comprises a contiguous nucleotide sequence complementary to at least 6 contiguous bases in the 3′ untranslated region (UTR) of an RNA sequence encoding glucocerebrosidase (GBA).

2. The antisense oligonucleotide according to claim 1, which is capable of increasing GBA expression.

3. The antisense oligonucleotide according to claim 1, which is capable of decreasing downregulation of GBA expression mediated by a microRNA (miR) in a target cell, wherein the miR is selected from miR-22-3p and a variant thereof comprising a seed region fully complementary to GGCAGCT.

4. The antisense oligonucleotide according to claim 1, wherein the antisense oligonucleotide is capable of inhibiting the binding of the RNA sequence to a miR selected from miR-22-3p and a variant thereof comprising a seed region fully complementary to GGCAGCT.

5. The antisense oligonucleotide according to claim 1, wherein the contiguous nucleotide sequence is 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40 nucleotides in length.

6. (canceled)

7. (canceled)

8. The antisense oligonucleotide according to claim 1, wherein the contiguous nucleotide sequence is at least 75%, at least 80%, at least 85%, at least 90%, at least 95%, or fully, complementary to contiguous bases located downstream of the stop codon TAG at positions 1746 to 1748 in SEQ ID NO:73.

9. (canceled)

10. The antisense oligonucleotide according to claim 1, wherein the contiguous nucleotide sequence is at least 75%, at least 80%, at least 85%, at least 90%, at least 95%, or fully, complementary to contiguous bases located in the segment at positions 2227 to 2274 in SEQ ID NO:73.

11. (canceled)

12. The antisense oligonucleotide according to claim 1, wherein the RNA sequence encoding GBA is an mRNA sequence comprising the sequence of SEQ ID NO:73 or an allelic variant thereof.

13. The antisense oligonucleotide according to claim 1, wherein the contiguous nucleotide sequence is complementary to a target nucleic acid sequence selected from the group consisting of SEQ ID NO:35, SEQ ID NO:36, SEQ ID NO:37, SEQ ID NO:38, SEQ ID NO:39, SEQ ID NO:40, SEQ ID NO:41, SEQ ID NO:42, SEQ ID NO:43, SEQ ID NO:44, SEQ ID NO:45, SEQ ID NO:46, SEQ ID NO:47, SEQ ID NO:48, SEQ ID NO:49, SEQ ID NO:50, SEQ ID NO:51, SEQ ID NO:52, SEQ ID NO:53, SEQ ID NO:54, SEQ ID NO:55, SEQ ID NO:56, SEQ ID NO:57, SEQ ID NO:58, SEQ ID NO:59, SEQ ID NO:60, SEQ ID NO:61, SEQ ID NO:62, SEQ ID NO:63, SEQ ID NO:64, SEQ ID NO:65, SEQ ID NO:66, SEQ ID NO:67, SEQ ID NO:68, or to a fragment of at least 10 nucleotides of any thereof.

14. The antisense oligonucleotide according to claim 1, wherein the antisense oligonucleotide is a single-stranded antisense oligonucleotide.

15. The antisense oligonucleotide according to claim 1, wherein the antisense oligonucleotide is a double-stranded antisense oligonucleotide.

16. The antisense oligonucleotide according to claim 1, wherein the contiguous nucleotide sequence has or comprises a nucleobase sequence selected from the group consisting of SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO:11, SEQ ID NO:12, SEQ ID NO:13, SEQ ID NO:14, SEQ ID NO:15, SEQ ID NO:16, SEQ ID NO:17, SEQ ID NO:18, SEQ ID NO:19, SEQ ID NO:20, SEQ ID NO:21, SEQ ID NO:22, SEQ ID NO:23, SEQ ID NO:24, SEQ ID NO:25, SEQ ID NO:26, SEQ ID NO:27, SEQ ID NO:28, SEQ ID NO:29, SEQ ID NO:30, SEQ ID NO:31, SEQ ID NO:32, SEQ ID NO:33, and SEQ ID NO:34, or a fragment of at least 10 nucleotides of any thereof.

17. (canceled)

18. The antisense oligonucleotide according to claim 1, wherein the antisense oligonucleotide comprises one or more modified nucleoside(s).

19. The antisense oligonucleotide according to claim 1, wherein the antisense oligonucleotide comprises one or more modified nucleoside(s) independently selected from the group consisting of 2′-O-alkyl-RNA, 2′-O-methyl-RNA, 2′-alkoxy-RNA, 2′-O-methoxyethyl-RNA, 2′-amino-DNA, 2′-fluoro-DNA, arabino nucleic acid (ANA), 2′-fluoro-ANA, morpholino, and locked nucleic acid (LNA) nucleosides.

20. (canceled)

21. (canceled)

22. (canceled)

23. The antisense oligonucleotide according to claim 1, wherein the antisense oligonucleotide is a mixmer or a totalmer, optionally wherein the mixmer does not comprise any DNA or RNA nucleosides.

24. The antisense oligonucleotide according to claim 1, which is a mixmer of 2′-MOE and LNA nucleosides or a totalmer of 2′-MOE nucleosides.

25. The antisense oligonucleotide according to claim 1, wherein the antisense oligonucleotide comprises at least one modified internucleoside linkage.

26. The antisense oligonucleotide according to claim 1, wherein the antisense oligonucleotide comprises one or more phosphorothioate internucleoside linkages.

27. (canceled)

28. The antisense oligonucleotide according to claim 1, wherein the antisense oligonucleotide is capable of increasing the expression of GBA by at least about 5%, at least about 10%, at least about 15%, at least about 20%, at least about 30%, at least about 40%, at least about 50%, or more than 50%, optionally compared to a control.

29. The antisense oligonucleotide according to claim 1, wherein the antisense oligonucleotide is covalently attached to at least one conjugate moiety.

30. The antisense oligonucleotide according to claim 1, wherein the antisense oligonucleotide is in the form of a pharmaceutically acceptable salt.

31. (canceled)

32. The antisense oligonucleotide according to claim 1, wherein the antisense oligonucleotide is encapsulated in a lipid-based delivery vehicle, covalently linked to or encapsulated in a dendrimer, or conjugated to an aptamer.

33. A pharmaceutical composition comprising the antisense oligonucleotide according to claim 1, and a pharmaceutically acceptable diluent, solvent, carrier, salt and/or adjuvant.

34. (canceled)

35. (canceled)

36. An in vitro or in vivo method for increasing or restoring GBA expression in a target cell, the method comprising administering an effective amount of the antisense oligonucleotide according to claim 1 to the target cell.

37. (canceled)

38. (canceled)

39. (canceled)

40. A method for treating or preventing a disease or disorder, comprising administering a therapeutically or prophylactically effective amount of the antisense oligonucleotide according to claim 1 to a subject suffering from or susceptible to a disease or disorder.

41. (canceled)

42. (canceled)

43. (canceled)

44. The method according to claim 40, wherein the disease is selected from the group consisting of Gaucher's disease, Parkinson's Disease, dementia, dementia with Lewy bodies (DLB) and rapid eye movements (REM) sleep behaviour disorders.

45. (canceled)

46. (canceled)
